(19) United States
(12) Patent Application Publication
(10) Pub. No.: US 2019/0054047 A1
(43) Pub. Date: Feb. 21, 2019

(54) TREATMENT OF MALIGNANT ADRENOCORTICAL TUMOR WITH NICLOSAMIDE AND OTHER COMPOUNDS

(71) Applicant: The U.S.A., as represented by the Secretary, Department of Health and Human Services, Bethesda, MD (US)

(72) Inventors: Electron Kebebew, Palo Alto, CA (US); Lisa Zhang, Rockville, MD (US); Ya-Qin Zhang, Rockville, MD (US); Min Shen, Boyds, MD (US)

(73) Assignee: The U.S.A., as represented by the Secretary, Department of Health and Human Services, Bethesda, MD (US)

(21) Appl. No.: 16/070,374

(22) PCT Filed: Jan. 18, 2017
(86) PCT No.: PCT/US2017/013873

§ 371 Date: Jul. 16, 2018

Related U.S. Application Data
(60) Provisional application No. 62/280,521, filed on Jan. 19, 2016.

(51) Int. Cl. A61K 31/167; A61K 31/03; A61P 35/00; A61K 38/12; A61K 31/4725; A61K 31/704; A61K 31/55; A61K 31/4995; A61K 31/305; A61K 31/706; A61K 31/7135; A61K 31/7048; A61K 31/555; A61K 33/24; A61K 31/357; A61K 31/7008

(52) U.S. Cl. CPC A61K 31/167; A61K 31/7008; A61P 35/00; A61K 38/12; A61K 31/4725; A61K 31/704; A61K 31/55; A61K 31/4995; A61K 31/305; A61K 31/706; A61K 31/7135; A61K 31/7048; A61K 31/555; A61K 33/24; A61K 31/357; A61K 31/03

(57) ABSTRACT

Disclosed herein are methods of treating a malignant adrenocortical tumor, including a locally advanced and metastatic adrenocortical carcinoma. In some examples, methods of treating a malignant adrenocortical tumor include administering an effective amount of niclosamide alone or in combination with other therapeutic agents to a subject in need thereof, thereby treating the malignant adrenocortical tumor.

### CROSS-REFERENCE TO RELATED APPLICATION

This application claims priority to U.S. Provisional Application No. 62/280,521 filed Jan. 19, 2016, herein incorporated by reference.

### FIELD

This disclosure is related to adrenocortical carcinoma, and in particular, to the use of niclosamide (or other agent listed in Table 1) for treatment of metastatic adrenocortical carcinoma.

### BACKGROUND

Adrenocortical carcinoma (ACC) is a highly aggressive endocrine cancer, with an incidence of one to two cases per million in the general population. Understanding of the molecular pathogenesis of ACC has improved, and alterations in CTNNB1, IGF-2, and TP53 occur in ACC cases and are associated with ACC prognosis. With a five-year overall survival rate ranging from 16% to 38%, the prognosis of patients with locally advanced and/or metastatic ACC is dismal. Surgical resection is the only available curative treatment, yet 60-80% of patients who undergo complete resection experience a recurrence. Mitotane and systemic chemotherapy with etoposide, doxorubicin, and cisplatin are used for patients with advanced or unresectable disease. Unfortunately, the available agents provide little clinical benefit for patients, leaving them with few treatment options. Given the limited success and significantly high toxicity of current drug regimens, there is an urgent need for new therapeutic options for patients with locally advanced and unresectable ACC.

### SUMMARY

Disclosed herein is the surprising discovery of the use of niclosamide therapy for treating locally advanced and metastatic adrenocortical carcinoma. High-throughput drug screening in adrenocortical carcinoma cell lines was performed using a library of 4,292 compounds. Niclosamide was identified and shown to induce durable anticancer activity in adrenocortical carcinoma in vitro and in vivo. Niclosamide induced caspase-dependent apoptosis and G1 arrest. Moreover, niclosamide treatment reduced β-catenin and mediators of epithelial-to-mesenchymal transition protein levels, and resulted in mitochondrial uncoupling, features omnipresent in human adrenocortical carcinoma samples. It is also shown herein that treatment with niclosamide in combination with mitotane provides a synergistic effect on tumor treatment in vitro.

Based upon these findings, provided herein are methods of treating adrenocortical carcinoma, and in particular, locally advanced and metastatic adrenocortical carcinoma. In some examples, a method of treatment includes administering to the subject an agent, such as a therapeutically effective amount of an agent listed in Table 1, thereby treating the malignant adrenocortical tumor. In some examples, the agent is niclosamide either alone or in combination with other therapeutic agents, such as those listed in Table 1. In one example, the additional therapeutic agent includes or consists of mitotane. In some examples, the method is used for treating a subject with ACC, such as locally advanced and metastatic ACC which was non-responsive to standard ACC therapies (such as surgery, radiotherapy, and/or chemotherapy (such as mitotane, cisplatin, doxorubicin, etoposide+mitotane or streptozotocin+mitotane).

The foregoing and other features of the disclosure will become more apparent from the following detailed description of a several embodiments which proceeds with reference to the accompanying figures.

### BRIEF DESCRIPTION OF THE DRAWINGS

FIG. 1 provides dose-response curves comparing niclosamide to cisplatin, doxorubicin, etoposide, mitotane, and streptozocin from qHTS.

FIGS. 2A-D illustrate the effect of niclosamide on cellular proliferation, three-dimensional multicellular aggregates, and apoptosis. FIG. 2A, Niclosamide inhibits cellular proliferation in a time- and dose-dependent manner in ACC cell lines. Error bars represent ±SD; **, P<0.01; ***, P<0.001; ****, P<0.0001. FIG. 2B, Three-dimensional multicellular aggregates (MCA) treated with niclosamide. FIG. 2C, Niclosamide treatment results in increased caspase 3/7 activity at 48 hours (SW-13) and 96 hours (NCI-H295R); **, P<0.01; ***, P<0.001; ****, P<0.0001. FIG. 2D, Niclosamide induces G₁ cell cycle arrest after 48 hours of treatment. Right panel shows percent of cells in each cell cycle phase.

FIGS. 3A and 3B illustrate niclosamide treatment results in mitochondrial uncoupling in ACC cells. FIG. 3A, Oxygen consumption rate (OCR) and extracellular acidification rate (ECAR) were measured using the Seahorse XF96 analyzer after niclosamide treatment (see Example 1, Materials and Methods). Point A (vertical lines) indicates injection of DMSO, niclosamide, or the positive control; **, P<0.01; ***, P<0.001; ****, P<0.0001. FIG. 3B, The effect of niclosamide on tetramethylrhodamine, ethyl ester (TMRE) in ACC cells. Cells treated for 3, 6, 18 or 24 hours with niclosamide were stained with TMRE to measure mitochondrial membrane potential. FCCP was used as a positive control; *, P<0.05; **, P<0.01; ***, P<0.001; ****, P<0.0001.

FIGS. 4A-4D illustrate the effect of niclosamide on β-catenin and cellular migration in ACC cells. FIG. 4A, Niclosamide reduces β-catenin in ACC cells. Western blot analysis after 48 hours and 72 hours of treatment with niclosamide. There was a dose-dependent decrease in β-catenin expression level. FIG. 4B, Cellular migration of NCI-H295R and SW-13 was assessed using a Boyden chamber assay. Cells were treated for 24 hours, trypsinized, and seeded in Boyden chambers, and allowed to migrate for 24 or 48 hours before fixation. Cells were counted in three random fields, and the experiment was performed in triplicate; ***, P<0.001; ****, P<0.0001. FIG. 4C, Reduced cellular migration of BD-140A as measured by the wound healing assay. FIG. 4D, Niclosamide reduces the expression levels of N-cadherin and vimentin. Representative Western blot analysis showing reduced expression of EMT markers, N-cadherin, and vimentin after 48 hours of treatment.

FIGS. 5A and 5B illustrate niclosamide treatment reduced ACC tumor growth in vivo. 5×10⁶ NCI-H295R cells were injected into the flank of Nu⁺/Nu⁺mice. Tumors were allowed to grow, and mice were randomized into three groups and treated as indicated. Tumor sizes (FIG. 5A) and mouse body weight (FIG. 5B) were measured weekly. Error bars are mean ±SEM for tumor volume, and mean ±STDEV for mouse body weight; *, P<0.05; **, P<0.01.

FIGS. 6A and 6B illustrate that niclosamide inhibits the Akt pathway, indicating that niclosamide acts on multiple cellular pathways.

FIGS. 7A-7D illustrate that niclosamide in combination with mitotane has a synergistic effects on inhibiting cellular proliferation as compared to niclosamide or mitotane treatment alone. Treatment with (A) mitotane alone, (B) niclosamide alone, or (C) both mitotane and niclosamide. (D) Combination effect was determined according to the Chou-Talalay method.

### DETAILED DESCRIPTION

### I. Introduction

The traditional drug-development process is costly and time consuming, with a high failure rate. It is estimated that it requires approximately $1 billion and 10 years to bring a drug to market. Drug repositioning or repurposing is an emerging field in which new applications are found for existing drugs. Drug repositioning has an advantage over de novo drug discovery because many drugs already have known pharmacokinetics, pharmacodynamics, and toxicity profiles, and this knowledge hastens the evaluation of the drug in clinical trials. For rare cancers such as ACC, drug repositioning can play an essential role in a disease that would otherwise be neglected due to high costs. Furthermore, drug repurposing may uncover new molecular pathways involved in carcinogenesis or reveal new molecular targets for therapy.

In this study, quantitative high-throughput screening (qHTS) was performed on 4,292 clinically approved compounds in three ACC cell lines. Twenty one compounds that were active in all cell lines were identified. One of the most potent compounds was niclosamide, an antihelminthic drug approved for human use for over 50 years. Niclosamide inhibited ACC cellular proliferation, induced caspase-dependent apoptosis and G₁ cell cycle arrest, and decreased ACC cellular migration. More importantly, niclosamide treatment dramatically inhibited ACC tumor growth in vivo with no observed side effects or toxicity in the mice. These findings indicate that niclosamide is an agent for the treatment of ACC.

This disclosure provides an effective strategy for identifying novel antineoplastic agents for ACC using qHTS. Of 4,292 compounds screened, 21 compounds had an efficacy >80% in all cell lines. Mechanistically, it was found that niclosamide induces caspase-dependent apoptosis and G₁ cell cycle arrest, and decreases cellular migration. Furthermore, it was determined that niclosamide is a potent mitochondrial uncoupler that inhibits cellular pathways involved in ACC. Further, the in vivo studies showed that niclosamide treatment greatly inhibited ACC xenograft tumor growth with no observed side effects or toxicity in mice, indicating that niclosamide can be used in in the clinic in ACC patients who do not respond standard therapies.

Niclosamide is an antihelminthic agent that has been approved by the United States Food and Drug Administration for the treatment of tapeworm infections in humans, and it has been in use for the past 50 years. This agent has a good safety profile and exhibits little toxicity even after long-term exposure. Niclosamide inhibits oxidative phosphorylation in the mitochondria of cestodes. In addition, niclosamide has antineoplastic activity in various cancers by inhibiting multiple cellular pathways known to play roles in carcinogenesis, including WNT/β-catenin, notch, mTOR, NF-kB, and STAT3 (Chen et al., Biochemistry. 2009; 48:10267-74; Wieland et al., Clin Cancer Res. 2013; 19:4124-36; Osada et al., Cancer Res. 2011; 71:4172-82; Jin et al., Cancer Res. 2010; 70:2516-27; You et al., Mol Cancer Ther. 2014; 13:606-16; Londono-Joshi et al., Mol Cancer Ther. 2014; 13:800-11; Arend et al., Gynecol Oncol. 2014; 134:112-20; Liu et al., Prostate. 2015; 75:1341-53; and King et al., Oncogene. 2015; 34:3452-62, each of which is hereby incorporated by reference in its entirety). The screening herein revealed that niclosamide has potent anticancer activity against multiple ACC cell lines, with an IC₅₀ that is well below the Cₘₐₓ, in both mice and humans. Niclosamide has low water solubility and oral bioavailability. These factors may result in a wide range of serum concentrations of niclosamide, which can result in variable anti-cancer efficacy. When compared to current drug treatments for ACC, niclosamide was found to have better activity than cisplatin, doxorubicin, etoposide, mitotane, or streptozocin, indicating that it is a viable novel agent that can be translated into clinical therapy for ACC patients who do not respond standard therapeutic regimens.

Validation of the screening results confirmed that niclosamide inhibits cellular proliferation in a time- and dose-dependent manner in both monolayer cell culture and MCA, and that it induces cell death at high doses. MCA has been widely considered to better reflect in vivo tumor growth due to similar volume of growth kinetics, proliferation gradients, and extracellular matrix production that supports cancer cell growth. The mechanism by which niclosamide inhibits cell proliferation was further characterized, demonstrating that it induces G₁ cell cycle arrest and caspase-dependent apoptosis.

The WNT/β-catenin pathway has been shown to play a role in ACC, with alteration of this pathway found in over 30% of ACC cases. In canonical WNT/β-catenin signaling, β-catenin complexes with adenomatous polyposis coli, axin, and glycogen synthase kinase-3b (GSK3b) are subsequently phosphorylated and degraded. In the presence of WNT, GSK3b inhibition results in the stabilization of β-catenin, which is then translocated into the nucleus and targets the expression of genes that regulate cell growth, motility, and differentiation. Mutations in β-catenin have been associated with poor prognosis in patients with ACC, and higher-grade ACC is associated with higher β-catenin expression. Silencing of β-catenin in NCI-H295R has been shown to decrease proliferation, induce cell cycle arrest and apoptosis, reverse the EMT phenotype, and inhibit in vivo tumor development. These findings indicate that the WNT/β-catenin pathway plays a major role in ACC tumorigenesis and may be an effective therapeutic target for ACC treatment. Despite the key role of WNT/β-catenin signaling in ACC, current treatments do not effectively target this pathway. Thus, niclosamide's ability to inhibit β-catenin expression may prove to be an exciting and effective new strategy for ACC treatment, especially given the large subset of patients with ACC who have alterations in the WNT/β-catenin pathway in their tumors.

Because niclosamide reduces β-catenin levels and plays a key role in the induction of EMT, whether niclosamide affects cellular migration was investigated. ACC is a highly invasive cancer with a high rate of metastasis. Cancer progression is associated with the loss of epithelial properties and the gain of mesenchymal characteristics, and this process is regulated by multiple proteins that mediate EMT in cancer progression. Consistent with the reduced level of β-catenin, decreased ACC cellular migration with niclosamide treatment was observed. Furthermore, this decrease was associated with reduced expression of the mesenchymal markers N-cadherin and vimentin. Thus, the observed reversal of the EMT phenotype may have important implications for the in vivo effects of niclosamide on ACC progression.

Niclosamide was found to act as a mitochondrial uncoupler in ACC cell lines. The mitochondrial dysregulation of cancer cells has been studied (Warburg effect; Warburg et al., Biochemische Zeitschrift. 1924; 152: 309-44). Cancer-specific changes in mitochondrial metabolism have been linked to malignant cell transformation, apoptosis evasion, the high proliferative capacity of cancer cells, and driver gene/pathway mutations. Mitochondrial uncouplers exert their effect by dissipating the proton gradient formed by the electron transport chain, thus uncoupling it from ATP production. While under the Warburg hypothesis many cancers appear to decrease their dependence on oxidative phosphorylation for ATP production, studies have shown that the ATP produced by oxidative phosphorylation may still be necessary to initiate glycolysis through hexokinase II activation. In addition, uncoupling oxidative phosphorylation has been associated with shifts in cancer cell metabolism, from the oxidation of pyruvate to the oxidation of glutamine and fatty acids. Studies on the antineoplastic effects of other mitochondrial uncouplers have demonstrated that they have an effect on cell cycle arrest and apoptosis. Furthermore, in a study on the effect of niclosamide in acute myelogenous leukemia, niclosamide was shown to cause mitochondrial damage, increase reactive oxygen species, and induce apoptosis through increased levels of cytochrome C (Cancer Res. 2010; 70:2516-27). The altered mitochondrial function seen in cancer cells may also explain the low toxicity of niclosamide in normal cells. The mitochondria of cancer cells can be hyperpolarized compared to normal cells allowing cationic compounds such as niclosamide will preferentially accumulate in cancer cells. In addition, the acidic environment produced by the high glycolytic rate in tumor cells may promote the availability and activity of the drug.

In summary, niclosamide and other agents in Table 1 were identified as novel antineoplastic agents for ACC. Niclosamide has antiproliferative and proapoptotic activities, and induces G₁ cell cycle arrest. In addition, niclosamide reduces β-catenin expression and acts as a potent mitochondrial uncoupler. Niclosamide greatly inhibited xenograft tumor growth in vivo with no toxicity in mice. These findings indicate that niclosamide has anticancer activity through the inhibition of multiple altered cellular pathways and cellular metabolism, and, thus, is a new agent for the treatment of patients with ACC, such as for those who do not respond to standard therapy.

### II. Terms

Unless otherwise noted, technical terms are used according to conventional usage. Definitions of common terms in molecular biology may be found in Benjamin Lewin, Genes IX, published by Jones and Bartlett Publishers, 2007 (ISBN 0763740632); Kendrew et al. (eds.), The Encyclopedia of Molecular Biology, published by Blackwell Science Inc., 1998; and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Administration: To provide or give a subject an agent, such as a therapeutic agent, by any effective route. Exemplary routes of administration include, but are not limited to, injection (such as subcutaneous, intramuscular, intradermal, intraperitoneal, intratumoral and intravenous), oral, intraductal, sublingual, rectal, transdermal, intranasal, and inhalation routes.

Adrenal tumor: Any benign or malignant neoplasms of the adrenal gland. Malignant adrenal tumors include neuroblastoma, ACC, and a minority of adrenal pheochromocytomas. Most adrenal pheochromocytomas and all adrenocortical adenomas are benign tumors, which do not metastasize or invade nearby tissues, but which may still cause significant health problems by giving rise to hormonal imbalances. Disclosed herein are methods of treating malignant adrenal tumors including ACC.

Adrenocortical carcinoma is an aggressive cancer originating in the cortex (steroid hormone-producing tissue) of the adrenal gland. Adrenocortical carcinoma is a rare tumor with an incidence of 1-2 per million population annually and accounts for 0.02-0.2% of all cancer deaths. Approximately half of all patients have metastatic disease at the time of diagnosis resulting in an average five-year survival of less than 10%. Currently there is limited knowledge regarding the initiation and pathophysiology of ACC.

Adrenocortical carcinoma is often associated with hormonal syndromes which can occur in patients with steroid hormone-producing (“functional”) tumors, including Cushing's syndrome, Conn syndrome, virilization and feminization. Due to their location deep in the retroperitneum, most adrenocortical carcinomas are not diagnosed until they have grown quite large. They frequently invade large vessels, such as the renal vein and inferior vena cava, as well as metastasizing via the lymphatics and through the blood to the lungs and other organs. Thus, metastatic disease or local invasion is the only absolute indicator of malignancy. Masses without these features are assessed preoperatively based on size, and imaging characteristics, although the findings of these studies often are unable to definitively categorize the tumor as benign or malignant. After resection, tumor pathology is assessed based on several histologic criteria including cell morphology, cellular proliferation, and tumor invasiveness (Weiss criteria). The only curative treatment is complete surgical excision of the tumor, which can be performed even in the case of invasion into large blood vessels, such as the renal vein or inferior vena cava. A large percentage of patients are not surgical candidates. Radiation therapy and radiofrequency ablation may be used for palliation in patients who are not surgical candidates.

Chemotherapy regimens typically include the drug mitotane, an inhibitor of steroid synthesis which is toxic to cells of the adrenal cortex, as well as standard cytotoxic drugs. One widely used regimen consists of cisplatin, doxorubicin, etoposide, and mitotane. The endocrine cell toxin streptozotocin has also been included in some treatment protocols. Chemotherapy may be given to patients with unresectable disease, to shrink the tumor prior to surgery (neoadjuvant chemotherapy), or in an attempt to eliminate microscopic residual disease after surgery (adjuvant chemotherapy).

Hormonal therapy with steroid synthesis inhibitors such as aminoglutethimide may be used in a palliative manner to reduce the symptoms of hormonal syndromes.

In contrast to malignant adrenal cortical tumors, adrenocortical adenomas are benign tumors of the adrenal cortex which are extremely common (present in 1-10% of persons at autopsy). The clinical significance of these neoplasms is twofold.

Most adrenocortical adenomas are less than 2 cm in greatest dimension and less than 50 g in weight. However, size and weight of the adrenal cortical tumors are no longer considered to be a reliable sign of benignity or malignancy. Grossly, adrenocortical adenomas are encapsulated, well-circumscribed, solitary tumors with solid, homogeneous yellow-cut surface. Necrosis and hemorrhage are rare findings. Pheochromocytoma is a neoplasm composed of cells similar to the chromaffin cells of the mature adrenal medulla. Pheochromocytomas occur in patients of all ages, and may be sporadic, or associated with a hereditary cancer syndrome, such as multiple endocrine neoplasia (MEN) types IIA and IID, neurofibromatosis type I, or von Rippel-Lindau syndrome. Only 10% of adrenal pheochromocytomas are malignant, while the rest are benign tumors. The most clinically important feature of pheochromocytomas is their tendency to produce large amounts of the catecholamine hormones epinephrine (adrenaline) and norepinephrine. This may lead to potentially life-threatening high blood pressure, or cardiac arrythmias, and numerous symptoms such as headache, palpitations, anxiety attacks, sweating, weight loss and tremor.

Diagnosis is often confirmed through urinary measurement of catecholamine metabolites. Typically, pheochromocytomas are initially treated with anti-adrenergic drugs to protect against catecholamine overload, with surgery employed to remove the tumor once the patient is medically stable.

Agent: Any protein, nucleic acid molecule (including chemically modified nucleic acids), compound, small molecule, organic compound, inorganic compound, or other molecule of interest. Agent can include a therapeutic agent, a diagnostic agent or a pharmaceutical agent. A therapeutic or pharmaceutical agent is one that alone or together with an additional compound induces the desired response (such as inducing a therapeutic or prophylactic effect when administered to a subject, including inhibiting or treating a malignant adrenocortical tumor, such as inhibiting or treating ACC). For example, a “therapeutic agent” is a chemical compound, small molecule, or other composition, such as an antisense compound, antibody, protease inhibitor, hormone, chemokine or cytokine, capable of inducing a desired therapeutic or prophylactic effect when properly administered to a subject. In some examples, the therapeutic agent includes niclosamide alone or in combination with another therapeutic agent, such as provided in Table 1.

Biological sample: A biological specimen containing genomic DNA, RNA (including mRNA and microRNA), protein, or combinations thereof, obtained from a subject. Examples include, but are not limited to, saliva, peripheral blood, urine, tissue biopsy, surgical specimen, and autopsy material . In one example, a sample includes a biopsy of an adrenal cortex, such as from a patient with a malignant or benign adrenocortical tumor or a healthy control subject. In other embodiments, the biological sample is blood, or a component thereof, such as plasma or serum.

Chemotherapeutic agents: Any chemical agent with therapeutic usefulness in the treatment of diseases characterized by abnormal cell growth. Such diseases include tumors, neoplasms, and cancer, including ACC. In some cases, a chemotherapeutic agent is a radioactive compound. One of skill in the art can readily identify a chemotherapeutic agent of use (e.g., see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer, L., Berkery, R. (eds): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993). Combination chemotherapy is the administration of more than one agent to treat cancer.

Contacting: Placement in direct physical association, including both a solid and liquid form. Contacting an agent with a cell can occur in vitro by adding the agent to isolated cells or in vivo by administering the agent to a subject.

Control: A “control” refers to a sample or standard used for comparison, for example for comparison with a test sample or treated subject. In one example a control is a tissue sample obtained from a patient (or plurality of patients) with a benign adrenocortical tumor. In some embodiments, the control is a sample obtained from a healthy patient (or plurality of patients) (also referred to herein as a “normal” control), such as a normal adrenocortical sample. In some embodiments, the control is a historical control or standard value (i.e. a previously tested control sample or group of samples that represent baseline or normal values, such as baseline or normal values in a benign adrenocortical tumor). In some examples the control is a standard value representing the average value (or average range of values) obtained from a plurality of patient samples (such as an average value or range of values from normal patients or patients that are believed to be “cancer free”—cancer is no longer detectable).

In some examples, a control is a test subject's condition prior to treatment, such as the size of a tumor prior to treatment. In some examples, a control is a subject's condition with ACC, such as the size of a tumor, but not treated with a niclosamide or other compound in Table 1.

Decrease: To reduce the quality, amount, or strength of something. In one example, a therapy decreases a tumor, such as ACC, such as a malignant adrenocortical tumor, (such as the size of a tumor, the volume of a tumor, the number of tumors, the metastasis of a tumor, or combinations thereof), or one or more symptoms associated with a tumor, for example as compared to the response in the absence of the therapy (such as a therapy administered to affect tumor size via administration of niclosamide or other compound(s) in Table 1). In a particular example, a therapy decreases the size of a tumor, the volume of a tumor, the number of tumors, the metastasis of a tumor, or combinations thereof, such as a decrease of at least 10%, at least 20%, at least 50%, at least 60%, at least 70%, at least 75%, at least 80%, at least 90%, or even at least 95%, as compared to the size of a tumor, the volume of a tumor, the number of tumors, the metastasis of a tumor, or combinations thereof prior to the treatment. In certain examples, a decrease is at least 2-fold, at least 3-fold, at least 4-fold, at least 5-fold, at least 6-fold, at least 8-fold, at least 10-fold, at least 15-fold, at least 20-fold, at least 30-fold or at least 40-fold, as compared to the size of a tumor, the volume of a tumor, the number of tumors, the metastasis of a tumor, or combinations thereof prior to the treatment. Such decreases can be measured using the methods disclosed herein.

Diagnosis: The process of identifying a disease by its signs, symptoms and/or results of various tests. The conclusion reached through that process is also called “a diagnosis.” Forms of testing commonly performed include blood tests, medical imaging, genetic analysis, urinalysis, and biopsy.

Effective amount: An amount of agent that is sufficient to generate a desired response, such as reducing or inhibiting one or more signs or symptoms associated with a condition or disease. When administered to a subject, a dosage will generally be used that will achieve target tissue concentrations. In some examples, an “effective amount” is one that treats one or more symptoms and/or underlying causes of any of a disorder or disease. In some examples, an “effective amount” is a therapeutically effective amount in which the agent alone with an additional therapeutic agent(s) (for example a chemotherapeutic agent), induces the desired response such as treatment of a tumor, such as a malignant adrenocortical tumor. In one example, a desired response is to decrease the size, volume, number, and/or metastasis of an ACC, such as a malignant adrenocortical tumor, in a subject to whom the therapy is administered. Complete tumor elimination is not required for the composition to be effective. For example, a composition can decrease tumor size, volume, number, and/or metastasis of an ACC, such as a malignant adrenocortical tumor, by for example by at least 20%, at least 50%, at least 60%, at least 70%, at least 80%, at least 90%, at least 95%, at least 98%, or even at least 100% (elimination of the tumor), as compared to tumor size, volume, number, and/or metastasis in the absence of the composition.

In particular examples, it is an amount of an agent effective to decrease a number of malignant adrenocortical carcinoma cells, such as in a subject to whom it is administered, for example a subject having one or more carcinomas. The cancer cells do not need to be completely eliminated for the composition to be effective. For example, a composition can decrease the number of cancer cells by a desired amount, for example by at least 20%, at least 50%, at least 60%, at least 70%, at least 80%, at least 90%, at least 95%, at least 98%, or even at least 100% (elimination of detectable cancer cells), as compared to the number of cancer cells in the absence of the composition.

In other examples, it is an amount of niclosamide capable of modulating a locally advanced and metastatic malignant adrenocortical tumor (such as associated with ACC) by least 20%, at least 50%, at least 60%, at least 70%, at least 80%, at least 90%, at least 95%, at least 98%, or even at least 100% (elimination of detectable tumor growth) by niclosamide.

Niclosamide: A teniacide in the anthelmintic family effective against cestodes, in particular tapeworms. It is a salicylanilide compound (5-chloro-N-(2-chloro-4-nitrophenyl)-2-hydrobenzamide) with the chemical formula of

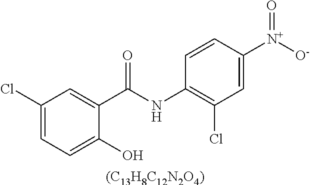

Niclosamide is also used as a piscicide. It is generally a chewable tablet taken orally. Disclosed herein is the use of niclosamide for treating ACC and in particular, locally advanced and metastatic ACC.

Patient or Subject: A term that includes human and non-human animals, such as those having an adrenocortical tumor, such as a malignant adrenocortical tumor. In one example, the patient or subject is a mammal, such as a human or veterinary subject (such as a dog, cat, mouse, or rat). “Patient” and “subject” are used interchangeably herein.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more therapeutic compounds, molecules or agents, such as those molecules listed in Table 1, including niclosamide.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Tissue: A plurality of functionally related cells. A tissue can be a suspension, a semi-solid, or solid. Tissue includes cells collected from a subject, such as from the adrenal cortex. A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Treating a disease: A phrase referring to a therapeutic intervention that ameliorates a sign or symptom of a disease or pathological condition after it has begun to develop.

Tumor, neoplasia, malignancy or cancer: The result of abnormal and uncontrolled growth of cells. Neoplasia, malignancy, cancer and tumor are often used interchangeably and refer to abnormal growth of a tissue or cells that results from excessive cell division. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” “Malignant cells” are those that have the properties of anaplasia invasion and metastasis.

Weiss criteria: A combination of the following nine criteria for distinguishing malignant adrenocortical tumors from benign adrenocortical tumors: nuclear grade III or IV; mitotic rate greater than 5/50 high-power fields; atypical mitoses; clear cells comprising 25% or less of the tumor; a diffuse architecture; microscopic necrosis; and invasion of venous, sinusoidal, and capsular structures. The presence of three or more of these features in a given tumor indicates malignant potential.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Methods of Treating a Malignant Adrenocortical Tumor

Provided herein is a method of treating a patient with a malignant adrenocortical tumor, including ACC, by administering to the patient a therapeutically effective amount of one or more agents listed in Table 1 (such as 1, 2, 3, 4 or 5 of the agents listed in Table 1) to reduce, inhibit, and/or prevent one or more signs or symptoms associated with the malignant adrenocortical tumor. In some embodiments, the one or more agents is niclosamide administered alone or in combination with other therapeutic agents, including one or more other agents listed in Table 1. In some examples, this method is used to treat a locally advanced and metastatic malignant adrenocortical tumor (such as associated with ACC).

Also disclosed are methods of inhibiting β-catenin expression. For example, a disclosed method includes contacting a cell, such as a malignant adrenocortical tumor cell, with an effective amount of one or more agents listed in Table 1, such as niclosamide alone or in combination with other agents, such as additional agents listed in Table 1, thereby inhibiting expression of β-catenin expression and thus, the signal transduction pathway regulated by β-catenin expression. In some examples, the cell is in vivo. In some examples, this method can be used to inhibit tumor growth, such as inhibiting the metastasis of ACC.

Also disclosed are methods of inhibiting or reducing the Akt pathway. For example, a disclosed method includes contacting a cell with an effective amount of one or more agents listed in Table 1, such as niclosamide alone or in combination with other agents, such as additional agents listed in Table 1, thereby inhibiting one or more molecules or activities of the Akt pathway. In some examples, this method can be used to inhibit tumor growth, such as inhibiting the metastasis of ACC.

In some examples, an “effective amount” is a therapeutically effective amount in which the agent alone, such as one or more agents listed in Table 1, such as niclosamide alone or in combination with other agents, such as additional agents listed in Table 1 or another chemotherapeutic agent(s) (such as mitotane), induces the desired response such as reduction of one or more signs or symptoms associated with a malignant adrenocortical tumor. In one example, a desired response is to decrease tumor size, tumor volume, tumor number, proliferation of a tumor, and/or metastasis (such as the size, volume and/or number of metastases) in a subject to whom the therapy is administered. A tumor does not need to be completely eliminated for the composition to be effective. For example, a composition can decrease the size, volume, proliferation and/or number of tumors by at least 20%, at least 50%, at least 60%, at least 70%, at least 80%, at least 90%, at least 95%, or at least 98%, such as a 20% to 90% decrease, a 50% to 80% decrease, a 60% to 80% decrease, a 60% to 90% decrease, such as a decrease of 20%, 25%, 30%, 35%, 40%, 45%, 50%, 55%, 60%, 65%, 70%, 75%, 80%, 85%, 90%, 95%, 97%, 98%, or even 100% (elimination of the tumor), as compared the size, volume, proliferation, and/or number of tumors in the absence of the therapy (e.g., prior to administration of the therapy). Similarly, tumor metastasis does not need to be completely eliminated for the composition to be effective. For example, a composition can decrease the size, volume and/or number of metastases by at least 20%, at least 50%, at least 60%, at least 70%, at least 80%, at least 90%, at least 95%, at least 98%, such as a 20% to 90% decrease, a 50% to 80% decrease, a 60% to 80% decrease, a 60% to 90% decrease, including 20%, 25%, 30%, 35%, 40%, 45%, 50%, 55%, 60%, 65%, 70%, 75%, 80%, 85%, 90%, 95%, 97%, 98%, or even 100% (elimination of the metastases), as compared to the size, volume and/or number metastasis in the absence of the composition (e.g., prior to administration of the therapy).

In particular examples, an “effective amount” is an amount of an agent effective to decrease a number of malignant adrenocortical carcinoma cells, such as in a subject to whom it is administered, for example a subject having one or more carcinomas. The cancer cells do not need to be completely eliminated for the composition to be effective. For example, a composition can decrease the number of cancer cells by at least 20%, at least 50%, at least 60%, at least 70%, at least 80%, at least 90%, at least 95%, at least 98%, such as a 20% to 90% decrease, a 50% to 80% decrease, a 60% to 80% decrease, a 60% to 90% decrease, including 20%, 25%, 30%, 35%, 40%, 45%, 50%, 55%, 60%, 65%, 70%, 75%, 80%, 85%, 90%, 95%, 97%, 98%, or even 100% (elimination of detectable cancer cells), as compared to the number of cancer cells in the absence of the composition.

In some examples, an effective amount of an agent, such as one or more agents listed in Table 1, such as niclosamide alone or in combination with other agents, such as additional agents listed in Table 1 or another chemotherapeutic agent(s) (such as mitotane), is that which reduces a sign or symptom associated with the malignant adrenocortical tumor by at least 10%, such as between 10% and 90%, between 10% and 50%, between 60% and 90%, including an about a 10%, 15%, 20%, 25%, 30%, 35%, 40%, 45%, 50%, 55%, 60%, 65%, 70%, 75%, 80%, 85%, 90%, 95% or 100% reduction (cancer is below the level of detection) as compared to the sign and/or symptom prior to the administration of the therapy.

In other examples, it is an amount of one or more agents listed in Table 1, such as niclosamide alone or in combination with other agents, such as additional agents listed in Table 1 or another chemotherapeutic agent(s) (such as mitotane), capable of modulating (reducing) a locally advanced and metastatic malignant adrenocortical tumor (such as associated with ACC) by least 20%, at least 50%, at least 60%, at least 70%, at least 80%, at least 90%, at least 95%, at least 98%, such as 20% to 90%, 50% to 80%, 60% to 80%, 60% to 90%, including 20%, 25%, 30%, 35%, 40%, 45%, 50%, 55%, 60%, 65%, 70%, 75%, 80%, 85%, 90%, 95%, 97%, 98%, or even 100% (elimination of detectable tumor growth) by niclosamide.

In some examples, the treatment methods include screening a subject for a malignant adrenocortical tumor, such as ACC, prior to administering a disclosed treatment. In particular examples, the subject is screened to determine if the adrenocortical tumor is malignant, indicating ACC, or benign. Examples of methods that can be used to screening for ACC include a combination of ultrasound, tissue biopsy, and serum blood levels. If blood or a fraction thereof (such as serum) is used, 1-100 μl of blood is collected. Serum can either be used directly or fractionated using filter cut-offs to remove high molecular weight proteins. If desired, the serum can be frozen and thawed before use. I f a tissue biopsy sample is used, 1-100 μg of tissue is obtained, for example using a fine needle aspirate. The biological sample (e.g., tissue biopsy or serum) is analyzed to determine expression of a biomarker associated with a malignant adrenocortical tumor (such as miR-483-3p or miR-483-5p and IGF2-mRNA), wherein the presence of such indicates that the tumor is malignant and further that it can be treated with the disclosed therapies. In some examples, the treatment methods include selecting a subject with a malignant adrenocortical tumor. In some examples, such subject is one that has been non-responsive to traditional therapies used to treat a malignant adrenocortical tumor. Thus, in some examples, the subject is one who has been previously treated with chemotherapy, radiotherapy, radiofrequency ablation, biologics (e.g., monoclonal antibodies), surgery, or combinations thereof, which has not been effective in treating the ACC (such as locally advanced and metastatic ACC). Thus, in some examples, the subject, prior to receiving treatment with one or more compounds in Table 1, such as niclosamide, has received treatment with mitotane, such as cisplatin, doxorubicin, etoposide+mitotane or streptozotocin+mitotane. In some examples, the subject, prior to receiving treatment with one or more compounds in Table 1, such as niclosamide, has received treatment with linsitinib (OSI-906).

Identification of subjects with the same medical condition, such as a malignant adrenocortical tumor, including ACC (such as locally advanced and metastatic ACC) and those that have been determined to be non-responsive to other therapies, can be accomplished by selecting all patients with the same diagnosis within electronic health records (EHR). EHRs are simply individual health records in a digitized format that can be accessed via a computer or computer-based system over a network. EHRs are designed to keep information about each encounter with the patient. For example, EHRs may include a person's health characteristics, medical history, past and current diagnoses, lab reports and results, x-rays, photographs, prescribed medication, billing and insurance information, contact information, demographics, and the like.

In some embodiments, the use further includes providing a second appropriate therapy for the subject with the malignant adrenocortical tumor; this second therapy can be administered concurrently with niclosamide, prior to, or after. In some examples, the second therapy includes or consists of a chemotherapeutic, such as mitotane.

Administration of agents

Agents (such as those listed in Table 1) can be administered to a subject in need of treatment using any suitable means known in the art. Methods of administration include, but are not limited to, intraductal, intradermal, intramuscular, intraperitoneal, parenteral, intravenous, intratumoral, subcutaneous, vaginal, rectal, intranasal, inhalation, or oral. Intranasal administration refers to delivery of the compositions into the nose and nasal passages through one or both of the nares and can comprise delivery by a spraying mechanism or droplet mechanism, or through aerosolization. Administration of the compositions by inhalant can be through the nose or mouth via delivery by spraying or droplet mechanisms. Delivery can be directly to any area of the respiratory system via intubation. Parenteral administration is generally achieved by injection. Injectables can be prepared in conventional forms, either as liquid solutions or suspensions, solid forms suitable for solution of suspension in liquid prior to injection, or as emulsions. Injection solutions and suspensions can be prepared from sterile powders, granules, and tablets. Administration can be systemic or local.

Agents can be administered in any suitable manner, for example with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines

Administration can be accomplished by single or multiple doses. The dose required can vary from subject to subject depending on the species, age, weight and general condition of the subject, the particular therapeutic agent being used and its mode of administration. An appropriate dose can be determined based upon the methods described herein.

In some examples, the agents are administered using an enteral or parenteral administration route. Suitable enteral administration routes include, for example, oral, rectal, or intranasal delivery. Suitable parenteral administration routes include, for example, intratumoral administration, intravascular administration (such as intravenous bolus injection, intravenous infusion, intra-arterial bolus injection, intra-arterial infusion and catheter instillation into the vasculature); subcutaneous injection or deposition, including subcutaneous infusion (such as by osmotic pumps); direct application to the tissue of interest, for example by a catheter or other placement device (e.g., a suppository or an implant comprising a porous, non-porous, or gelatinous material); and inhalation.

In one example oral administration is used. In some examples, one or more compounds from Table 1 is administered orally to a subject with ACC. For examples, the one or more compounds is administered orally using a number of different techniques. In one example, niclosamide alone or in combination with other therapeutic agents (such as those disclosed in Table 1) is provided by oral gavage. In some examples, gavage can be undertaken using rigid dosing cannulae, flexible catheters or tubes can be used. As an alternative to gavage, some materials may be consumed voluntarily in palatable mixtures. Material can also be dosed using a small flexible catheter introduced only into the subject's mouth.

Exemplary doses include milligram or microgram amounts of the one or more agents in Table 1, such as niclosamide, per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). In some examples, a subject is administered at least 1 μg/kg of a compound in Table 1 daily, such as at least 10 μg/kg, at least 50 μg/kg, at least 100 μg/kg, at least 500 μg/kg, at least 1 mg/kg, at least 2 mg/kg, at least 5 mg/kg, at least 10 mg/kg, at least 25 mg/kg, at least 50 mg/kg, at least 100 mg/kg, at least 200 mg/kg, at least 300 mg/kg, at least 400 mg/kg, at least 50 mg/kg, at least 750 mg/kg, at least 1 g/kg, at least 2 g/kg, or at least 5 g/kg of a compound in Table 1 daily.

In some examples, a subject is orally administered at least 10 mg/kg of niclosamide daily, such as at least 25 mg/kg, at least 50 mg/kg, at least 100 mg/kg, at least 200 mg/kg, at least 300 mg/kg, at least 400 mg/kg, at least 50 mg/kg, at least 750 mg/kg, or at least 1 g/kg niclosamide daily. In some examples, a subject in need thereof is administered between 100 mg/kg of niclosamide and 400 mg/kg of niclosamide everyday by oral administration. In some examples, a subject is orally administered between 100 mg/kg of niclosamide and 200 mg/kg of niclosamide every day. In some examples, a subject in need thereof is administered between 100 mg/kg of niclosamide and 400 mg/kg of niclosamide everyday by oral administration.

In some examples, in addition to treatment with niclosamide (or other agent in Table 1), the subject also receives an effective amount of mitotane orally, for example at a dose of at least 0.5 g per day, at least 1 g per day, at least 2 g per day, at least 3 g per day, at least 4 g per day, at least 5 g per day, at least 6 g per day, at least 7 g per day, at least 8 g per day, at least 9 g per day, at least 10 g per day, at least 11 g per day, at least 12 g per day, at least 13 g per day, at least 14 g per day, at least 15 g per day, or at least 16 g per day, such as 2 to 6 g per day, 9 to 10 g per day, 2 g per day, 3 g per day, 4 g per day, 5 g per day, 6 g per day, 7 g per day, 8, g per day, 9 g per day, 10 g per day, 11 g per day, 12 g per day, 13 g per day, 14 g per day, 15 g per day, 16 g per day 17 g per day, 18 g per day or 19 g per day. Such doses can be divided in 2, 3 or 4 doses over the day. In some examples, mitotane is administered to achieve a blood concentration of 14 to 20 mg/L.

In some examples, in addition to treatment with niclosamide (or other agent in Table 1), the subject also receives an effective amount of cisplatin iv, for example at a dose of at least 50 mg/m² IV per cycle, at least 60 mg/m² IV per cycle, at least 70 mg/m² IV per cycle, at least 75 mg/m² IV per cycle, or at least 100 mg/m² IV per cycle, such as once every 3-4 weeks. In some examples, the subject receives 50 mg/m² IV per cycle, 60 mg/m² IV per cycle, 70 mg/m² IV per cycle, 75 mg/m² IV per cycle, or 100 mg/m² IV per cycle, such as once every 3-4 weeks.

In some examples, in addition to treatment with niclosamide (or other agent in Table 1), the subject also receives an effective amount of doxorubicin iv, for example at a dose of at least 10 mg/m² IV, at least 20 mg/m² IV, at least 40 mg/m² IV, at least 50 mg/m² IV, at least 60 mg/m² IV, or at least 75 mg/m² IV, such as once every 21 to 28 days. In some examples, the subject receives 40 to 60 mg/m² IV every 21 to 28 days, or 60 to 75 mg/m² IV once every 21 days.

In some examples, in addition to treatment with niclosamide (or other agent in Table 1), the subject also receives an effective amount of etoposide orally or via iv. For example, etoposide can be administered at a dose of at least 10 mg/m² IV, at least 20 mg/m² IV, at least 35 mg/m² IV, at least 50 mg/m² IV, at least 70 mg/m² IV, or at least 100 mg/m² IV on days 1 through 4 or 5, such as 35 mg/m² IV or 50 to 100 mg/m² IV days 1 through 4 or 5 or on days 5-7 (for example if doxorubicin and cisplatin are also part of the therapy). In some examples, the subject then receives at least 10 mg/m² IV, at least 20 mg/m² IV, at least 35 mg/m² IV, at least 50 mg/m² IV, at least 70 mg/m² IV, or at least 100 mg/m² IV for 4 or 5 days, such as 35 mg/m² IV or 50 to 100 mg/m² IV for 4 or 5 days. The oral dose for etopside is twice the IV dose rounded to the nearest 50 mg and given in 2 divided doses if greater than 400 mg.

In some examples, in addition to treatment with niclosamide (or other agent in Table 1), the subject also receives an effective amount of streptozotocin iv, such as at least 100 mg/m²/day, at least 200 mg/m²/day, at least 300 mg/m²/day, at least 400 mg/m²/day, or at least mg/m²/day, for example for 5 days, repeated every 4-6 weeks.

In some examples, in addition to treatment with niclosamide (or other agent in Table 1), the subject also receives treatment with cisplatin, doxorubicin, etoposide, and mitotane, for example at the dosages described above. In some examples, in addition to treatment with niclosamide (or other agent in Table 1), the subject also receives treatment with streptozotocin and mitotane, for example at the dosages described above.

### Combination Treatment Methods

The disclosed methods for inhibiting or treating malignant adrenocortical tumors can be used alone or can be accompanied by administration of other anti-cancer agents or therapeutic treatments (such as surgical resection of a tumor or radiation therapy). Any suitable anti-cancer agent can be administered to a patient as part of a treatment regimen that includes inhibiting or treating a malignant adrenocortical tumor. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g. anti-androgens) and anti-angiogenesis agents. Other anti-cancer treatments include radiation therapy and immuno therapy (such as antibodies). Such additional treatments can be provided before, during (e.g., concurrently) or after treatment with one or more agents in Table 1 (e.g., niclosamide).

Thus, in one example, the methods include treatment with niclosamide and one or more additional anti-cancer agents, such as a chemotherapeutic agent. In one example, the additional treatment includes or consists of mitotane. In one example, the additional treatment includes or consists of a PD-1 inhibitor (e.g., nivolumab, pembrolizumab, durvalumab, and atezolizumab).

Examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine).

Examples of antimetabolites include folic acid analogs (such as methotrexate), pyrimidine analogs (such as 5-FU or cytarabine), and purine analogs, such as mercaptopurine or thioguanine.

Examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase).

Other exemplary agents include platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide).

Examples of hormones and antagonists that can be used include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone).

Other exemplary chemotherapy drugs include Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen

Mustard, Taxol (or other taxanes, such as docetaxel), Velban, Vincristine, VP-16, while some more newer drugs include Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol.

In some examples, the chemotherapy regimen includes mitotane (an inhibitor of steroid synthesis which is toxic to cells of the adrenal cortex) as well as standard cytotoxic drugs. For example, an exemplary regimen can include one or more compounds in Table 1 (such as niclosamide) in combination with cisplatin, doxorubicin, etoposide, and mitotane. In some examples, the endocrine cell toxin streptozotocin is included. In further examples, hormonal therapy with steroid synthesis inhibitors such as aminoglutethimide is used in a palliative manner to reduce the symptoms of hormonal syndromes associated with the ACC.

In some examples, one or more compounds in Table 1 (such as niclosamide) is used in combination with a one or more biologics, such as a therapeutic antibody, such as a monoclonal antibody. Examples of such biologics that can be used include one or more of bevacizumab, cetuximab, panitumumab, pertuzumab, trastuzumab, bevacizumab (Avastin®), ramucirumab, and the like. In specific examples, the antibody or small molecules used as part of the therapy include one or more of the monoclonal antibodies cetuximab, panitumumab, pertuzumab, trastuzumab, bevacizumab (Avastin®), ramucirumab, or a small molecule inhibitor such as gefitinib, erlotinib, and lapatinib.

In some examples, one or more compounds in Table 1 (such as niclosamide) is used in combination with an agent that targets IGF1R, such as IMC-A12 (a monoclonal antibody) or OSI-906, a small molecule IGF1R antagonist.

In some examples, one or more compounds in Table 1 (such as niclosamide) is used in combination with an immunotherapy, such as a checkpoint inhibitor, such as those that target PD-1 or PDL-1 (e.g., nivolumab, pembrolizumab, durvalumab, atezolizumab, avelumab, BMS-936559, and MPDL3280A), CTLA-4 (e.g., tremelimumab and ipilimumab), B7-H3 (e.g., MGA271), and CD27 (e.g., CDX-1127).

In some examples, the subject receiving the one or more compounds in Table 1 (such as niclosamide) is also administered interleukin-2 (IL-2), as part of the therapy, for example via intravenous administration. In particular examples, IL-2 is administered at a dose of at least 500,000 IU/kg as an intravenous bolus over a 15 minute period every eight hours beginning on the day after administration of the one or more compounds in Table 1 (such as niclosamide) and continuing for up to 5 days. Doses can be skipped depending on subject tolerance.

In some examples, the subject receiving the one or more compounds in Table 1 (such as niclosamide) is also administered a fully human antibody to cytotoxic T-lymphocyte antigen-4 (anti-CTLA-4) as part of the therapy, for example via intravenous administration. In some example subjects receive at least 1 mg/kg anti-CTLA-4 (such as 3 mg/kg every 3 weeks or 3 mg/kg as the initial dose with subsequent doses reduced to 1 mg/kg every 3 weeks).

When used in combination with the administration of one of the disclosed therapeutic agents, the additional treatment methods described herein can be administered or performed prior to, at the same time, or following the disclosed anti-tumor therapy as appropriate for the particular patient, the additional symptoms associated with the ACC (e.g., hormonal symptoms, conditions and related diseases) and the specific combination of therapies.

The following examples are provided to illustrate certain particular features and/or embodiments. These examples should not be construed to limit the disclosure to the particular features or embodiments described.

### EXAMPLE 1

### Materials and Methods

This is example describes the materials and methods used to perform the studies in Examples disclosed herein.

### Cell Culture

NCI-H295R and SW-13 cells were cultured in Dulbecco's modified Eagle medium (DMEM) supplemented with 2.5% NuSerum (BD Biosciences, San Jose, Calif.) and 0.1% ITS premix (BD Biosciences, San Jose, Calif.). BD140A cells, provided by Drs. Kimberly Bussey and Michael Demeure (Phoenix, Ariz.), were cultured in RPMI supplemented with 1% L-glutamate (Gibco, Grand Island, N.Y.), 1% penicillin-streptomycin (Gibco, Grand Island, N.Y.), and 10% fetal bovine serum (FBS; Invitrogen, Carlsbad, Calif.). The cell lines were authenticated using short tandem repeat profiling. Cells were maintained in a 5% CO₂ atmosphere at 37° C.

qHTS Screening

The National Institutes of Health Chemical Genomic Center pharmaceutical library consists of 4,292 small molecules and compounds. Compounds were prepared as described previously (Mehta et al., Clin Cancer Res. 21:4123-32, 2015; hereby incorporated by reference in its entirety). The cell viability of treated cells was measured using the luciferase-coupled ATP quantitation assay, Cell Titer-Glo (Promega, Madison, Wis.). Doxorubicin hydrochloride and tetraoctylammonium bromide were used as positive controls. The final concentration of compounds in the assay ranged from 0.6 nM to 46 μM. The titration-response data was plotted and modeled by a four-parameter logistic fit-to-yield half-maximal inhibitory concentration (IC₅₀) and efficacy (maximal response), as compared to tetraoctylammonium bromide values.

### Reagents

The following antibodies were used: anti-beta catenin (1:1,000) from R&D Systems (Minneapolis, Minn.); anti-GAPDH (1:3,000) from Santa Cruz Biotechnology (Dallas, Tex.); anti-N-cadherin (1:4,000) from EMD Millipore (Billerica, Mass.); and anti-vimentin (1:5,000) from Cell Signaling Technology (Boston, Mass.). Niclosamide (2′,5-dichloro-4′-nitrosalicylanilide) was purchased from Sigma-Aldrich (St. Louis, Mo.) and dissolved in DMSO for in vitro studies.

### Cellular Proliferation Assay

3×10³ and 6×10³ cells were plated in 96-well plates depending on the cell line. 100 μL of fresh culture medium containing the drug or vehicle was added. Cell count was determined using the CyQuant kit (Life Technologies, Grand Island, N.Y.), according to the manufacturer's instructions, and cell number was measured using a SpectraMax M5 microplate reader (ex485/em538; Molecular Devices, Sunnyvale, Calif.). Assays were performed in quadruplicate and the experiments were repeated three times.

NCI-H295R and SW-13 cells, which form multicellular aggregates (MCA) or spheroids, were plated in Ultra Low Cluster 24-well plates (Costar, Corning, N.Y.) at 1×10⁵ cells/0.5 mL or 6×10⁴ cells/0.5 mL depending on the cell line. Spheroids were allowed to develop for one or two weeks at 37° C. in 5% CO_{2,} and media was exchanged twice a week. Spheroids were treated with niclosamide or the vehicle at varying concentrations, and imaged weekly.

### Caspase 3/7 Activity Assay

Cells were plated in 96-well plates and treated with niclosamide or the vehicle. Caspase 3/7 activity was measured using the Caspase-Glo 3/7 assay (Promega, Madison, Wis.), according to manufacturer's instructions.

### Cell Cycle Analysis

Cells plated in six-well plates were treated with niclosamide or the vehicle. At 48 hours, cells were fixed for 30 minutes in 70% ethanol at 4° C., and stained with 50 μg/mL of propidium iodide containing 100 mg/mL of ribonuclease A. Flow cytometry was performed on a Canto I flow cytometer (Becton-Dickinson, Franklin Lakes, N.J.) using CellQuest software (BD Biosciences, San Jose, Calif.). Data was generated for at least 20,000 events per sample and analyzed using Modfit software (Verity Software House, Inc., Topsham, Me.).

### Cellular Migration Assays

NCI-H295R and SW-13 cells were plated in six-well plates and treated with varying concentrations of niclosamide or the vehicle for 24 hours. Cells were trypsinized and plated in transwell chambers (BD Biosciences, San Jose, Calif.) at a density of 1×10⁵ cells per 0.5 mL. The lower chamber was filled with DMEM supplemented with 10% FBS as a chemoattractant. Cells were allowed to migrate for 24 hours or 48 hours depending on the cell line, and were fixed and stained with Diff-Quik (Dade Behring, Newark, N.J.). Cells were imaged and counted in three random fields per well, and the experiments performed in triplicate. For the wound-healing assay in BD140A cells, which do not migrate in the Boyden chamber model, cells were plated in six-well plates until confluent and treated with niclosamide or the vehicle. The cells were scratched using a sterile pipette tip and photographed at various time points.

### Western Blot Analysis

Cell lysates were analyzed by SDS-PAGE and transferred to a PVDF membrane. The membranes were incubated with the appropriate primary antibodies overnight at 4° C., followed by horseradish peroxidase conjugated IgG (anti-rabbit 1:3,000, Cell Signaling Technology; anti-mouse 1:3,000, Santa Cruz Biotechnology; anti-goat 1:2,000, R&D Systems, Inc.). Proteins were detected by enhanced chemiluminescence (ECL; Thermoscientific, Rockford, Ill.).

### Mitochondrial Metabolism Assays

The Seahorse XF96 assay was performed according to the manufacturer's instructions (Seahorse Bioscience, Billerica, Mass.). Briefly, cells were plated in XF96 plates and media was replaced with unbuffered DMEM supplemented with 2 mmol of glutamine and 2.5 g/L of D-Glucose. After 1 hour in a 37° C. CO₂-free incubator, the oxygen consumption rate (OCR) and extracellular acidification rate (ECAR) were measured before and after injection of the drug, the vehicle, or carbonyl cyanide 4-(trifluoromethoxy) phenylhydrazone (FCCP) using the Seahorse XF96 extracellular flux analyzer.

Cells in 96-well plates were treated with the drug or vehicle. Cells were then stained with 200 nM of tetramethylrhodamine, ethyl ester (TMRE) for 20 minutes at 37° C. and were washed with PBS; fluorescence was measured using a SpectraMax M5e 96-well fluorescence microplate reader. FCCP was used as a positive control.

### In Vivo Mouse Studies

Mice were maintained according to National Institutes of Health (NIH) Animal Research Advisory Committee (ARAC) guidelines. 5×10⁶ NCI-H295R cells were injected into the flank of Nu⁺/Nu⁺mice. Tumors were allowed to grow and mice were randomized into three treatment groups (8 mice per treatment group). M ice were treated with 100 mg/kg of niclosamide, 200 mg/kg of niclosamide, or the vehicle (PEG500) everyday by oral gavage. Tumor sizes were measured in two dimensions every week with calipers and recorded.

### Statistical Analysis

Statistical analysis was performed using GraphPad Prism 6 software (GraphPad Software, La Jolla, Calif.). Data were analyzed using a two-tailed t-test or Mann-Whitney test. Statistical significance was defined as a P-value of less than 0.05. Data are presented as mean±standard deviation (SD) or mean±standard error of the mean (SEM).

### EXAMPLE 2

### Screening of Compounds Treatment of ACC

A screening of 4,292 compounds was performed on three ACC cell lines: BD140A, SW-13, and NCI-H295R. Twenty-one active compounds were identified, with an efficacy of >80% in all three cell lines. Of these, niclosamide showed higher efficacy and lower IC₅₀ than established anti-ACC drugs. Niclosamide-inhibited cellular proliferation in all three ACC cell lines was then validated.

The mechanism by which niclosamide inhibited ACC cell proliferation was determined, and it was observed that it induced caspase-dependent apoptosis and G1 cell cycle arrest. Niclosamide also decreased cellular migration and reduced the level of mediators of epithelial-to-mesenchymal transition, such as N-cadherin and vimentin. Furthermore, niclosamide treatment resulted in decreased expression of β-catenin. The effect of niclosamide on energy metabolism in ACC cell lines was evaluated and it was found to result in mitochondrial uncoupling. Niclosamide treatment inhibited ACC tumor growth with no observed toxicity in mice in vivo.

These studies indicate that niclosamide has anti-ACC activity through its inhibition of multiple altered cellular pathways and cellular metabolism in ACC.

qHTS Identifies Niclosamide as an Active Agent Against ACC Cell Lines

qHTS was performed to identify compounds with anti-ACC activity in BD140A, SW-13, and NCI-H295R cells. 21 compounds were found to be pan-active in all cell lines, with an efficacy >80% (Table 1).

TABLE 1
Compounds pan-active in adrenocortical carcinoma cell lines NCIH295R,
BD140A and SW-13.
Compound Name

4-Chloromercuriphenol
Aclarubicin hydrochloride
Actinomycin
D; Dactinomycin; NSC-3053
alpha-Tomatine
Auranofin
Carminomycin
Chromomycin A3
Deslanoside
Digitoxin
Digoxin
Emetine
Idarubicin hydrochloride
Lanatoside A
Lanatoside C
Niclosamide
o-(Chloromercuri)phenol
Omacetaxine mepesuccinate
Ouabain
Plicamycin
Trabectedin
Zinc pyrithione

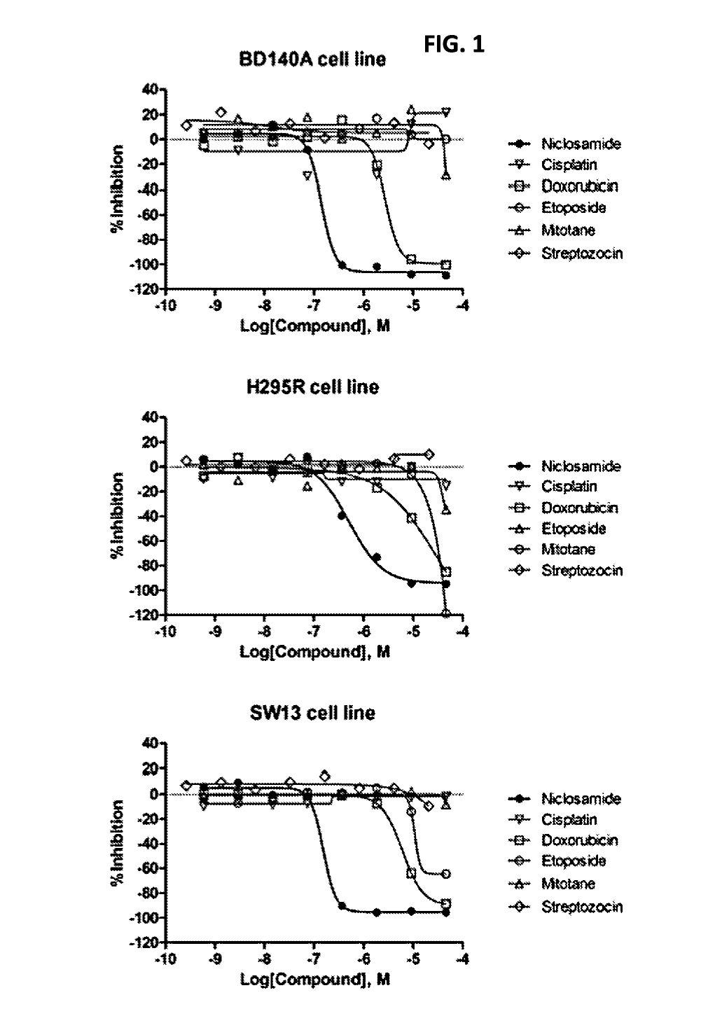

One of the highly active agents identified was niclosamide, which had an IC₅₀ of 0.12 μM, 0.15 μM, and 0.53 μM in BD140A, SW-13, and NCI-H295R, respectively; these values are well below the known Cₘₐₓ in humans of 18.34 μM. A comparison of niclosamide to commonly used drugs for ACC showed that niclosamide had better activity (lower IC₅₀ and higher efficacy) compared to cisplatin, doxorubicin, etoposide, mitotane, and streptozocin (FIG. 1).

### EXAMPLE 3

### Niclosamide Inhibits Cellular Proliferation and Induces Caspase-Dependent Apoptosis

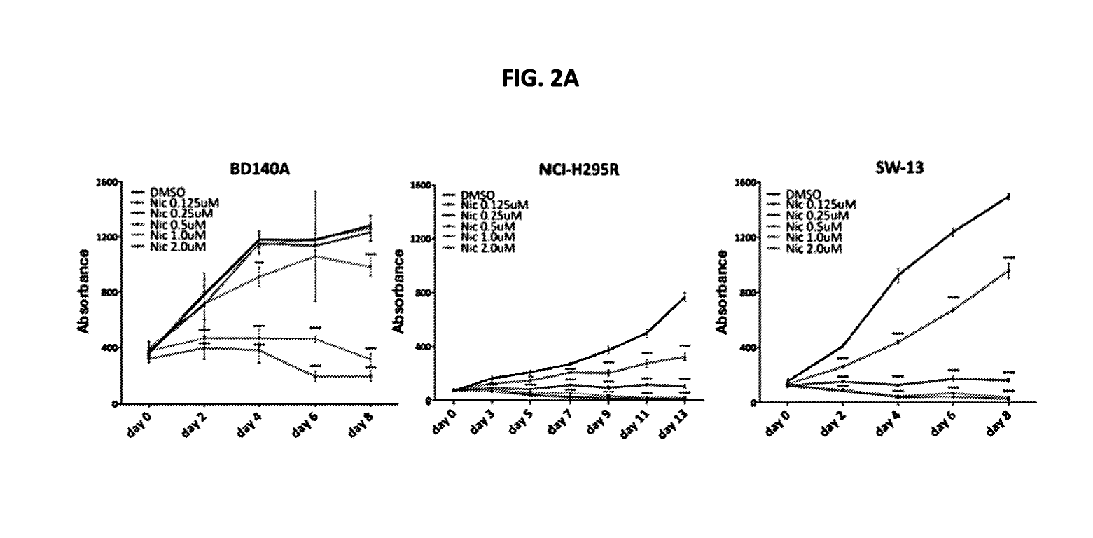

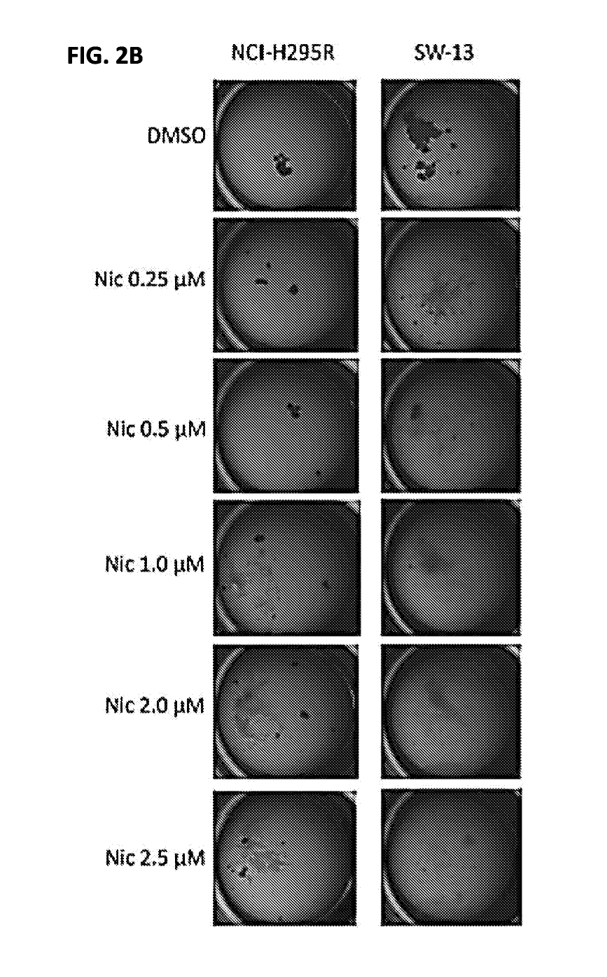

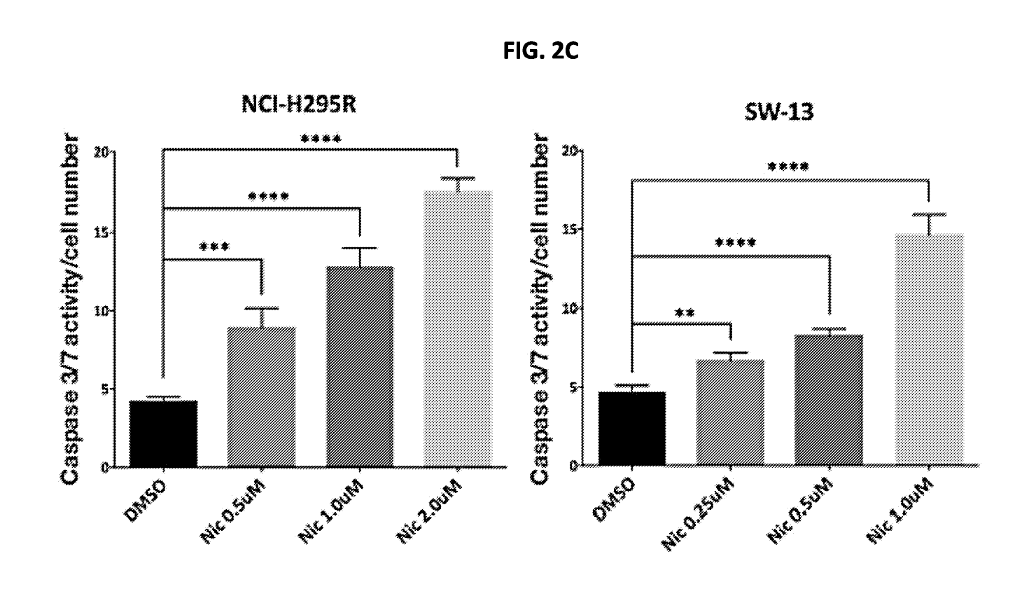

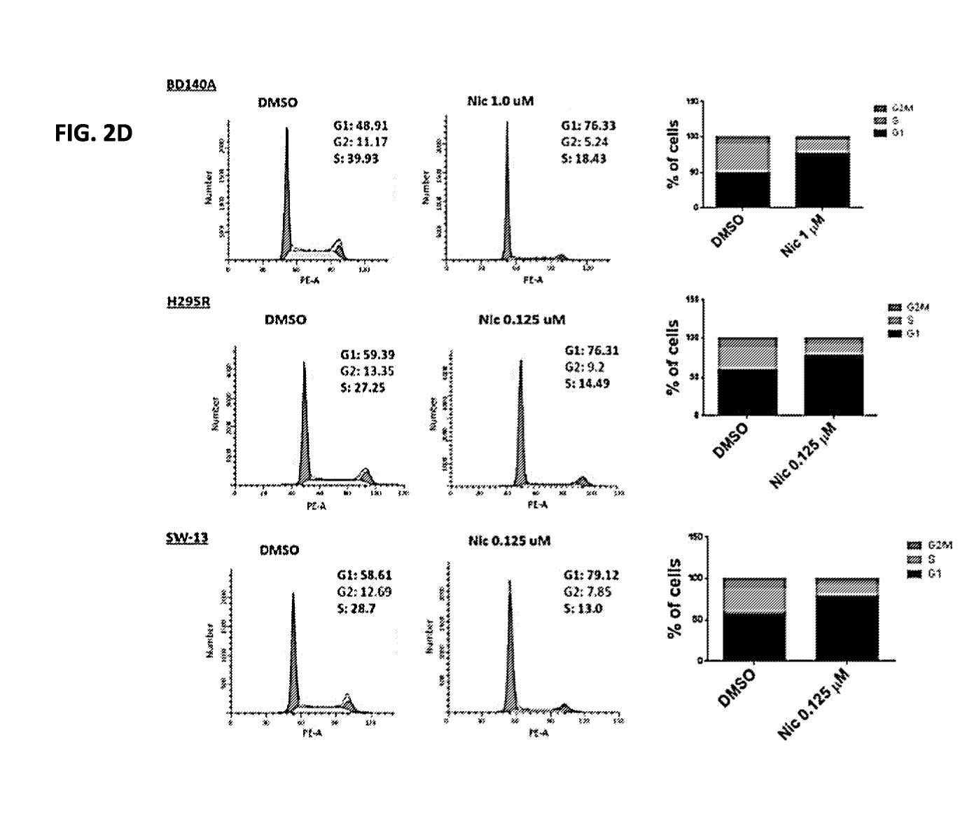

The antiproliferative effects of niclosamide were validated in the three ACC cell lines. The effect of niclosamide was first evaluated in a monolayer cell culture. Niclosamide inhibited proliferation in a time- and dose-dependent manner (FIG. 2A). Also, at higher concentrations, niclosamide treatment was cytotoxic, killing pre-existing cancer cells (FIG. 2A). The antiproliferative activity of niclosamide was assessed in the cell lines (SW-13 and NCI-H295R) that form MCAs. After three and four weeks of treatment of SW-13 and NCI-H295R, respectively, growth inhibition and disintegration of the MCAs was observed (FIG. 2B).

To further elucidate the mechanism by which niclosamide inhibited cellular proliferation and caused cell death, the effect on cell cycle progression and apoptosis was determined. Niclosamide treatment of NCI-H295R and SW-13 cell lines increased caspase 3/7 activity at 48 hours and 96 hours, respectively (FIG. 2C). However, no increase in caspase 3/7 activity was observed in BD140A cells. In contrast, niclosamide treatment induced G₁ cell cycle arrest, with an observed increase in the number of cells in the G₁ phase and a decrease in the number of cells in the S phase in all three ACC cell lines (FIG. 2D).

### EXAMPLE 4

### Niclosamide Results in Mitochondrial Uncoupling

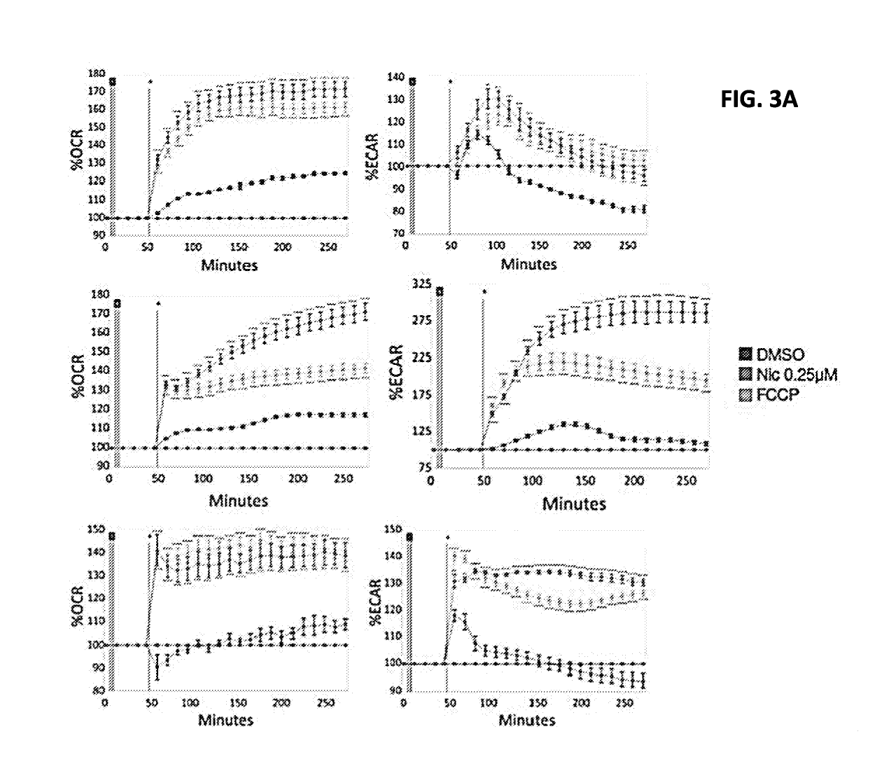

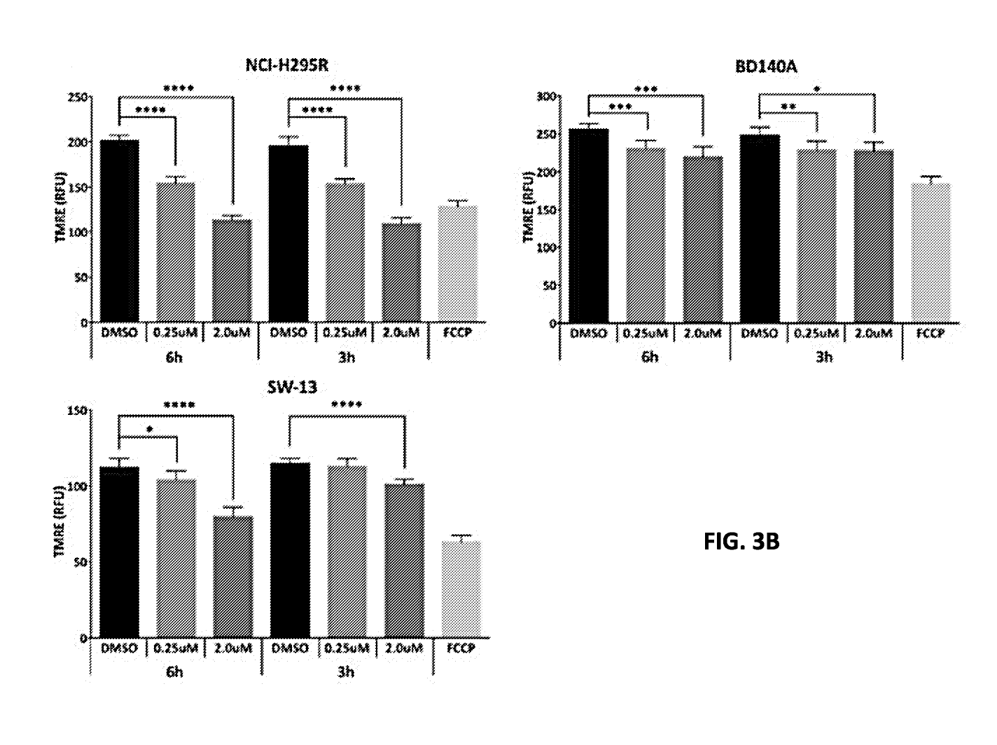

Niclosamide's antiparasitic mechanism of action has been reported to be due to the uncoupling of oxidative phosphorylation. To determine whether niclosamide has an uncoupling effect in ACC cell lines, the OCR and ECAR were measured using the Seahorse XF96 analyzer. The OCR and ECAR increased with niclosamide treatment in all three cell lines, indicating an uncoupling of the electron transport chain from ATP synthesis and the subsequent metabolic shift to glycolysis for energy production (FIG. 3A). Staining of niclosamide-treated ACC cells with

TMRE confirmed a decrease in mitochondrial membrane potential after 3 hours and 6 hours of niclosamide treatment (FIG. 3B).

### EXAMPLE 5

Niclosamide Reduces the Expression of β-Catenin, and Decreases Cellular Migration and Mediators of Epithelial-to-Mesenchymal Transition

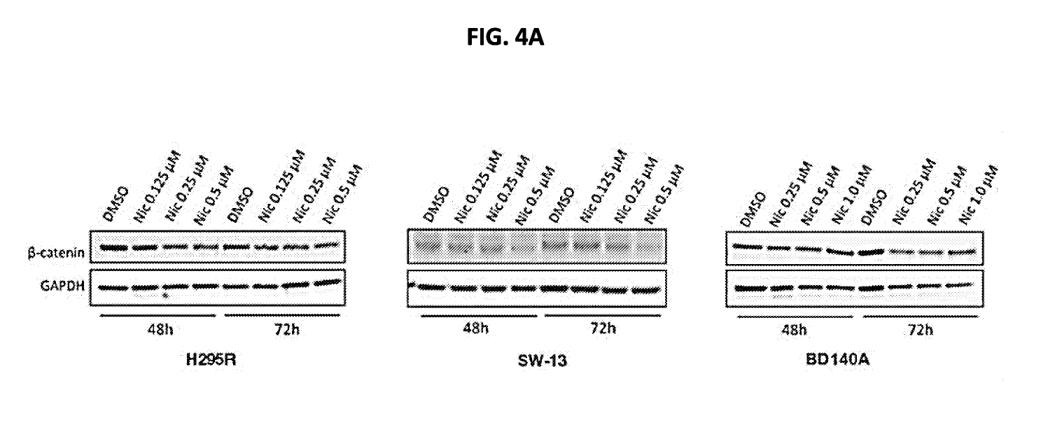

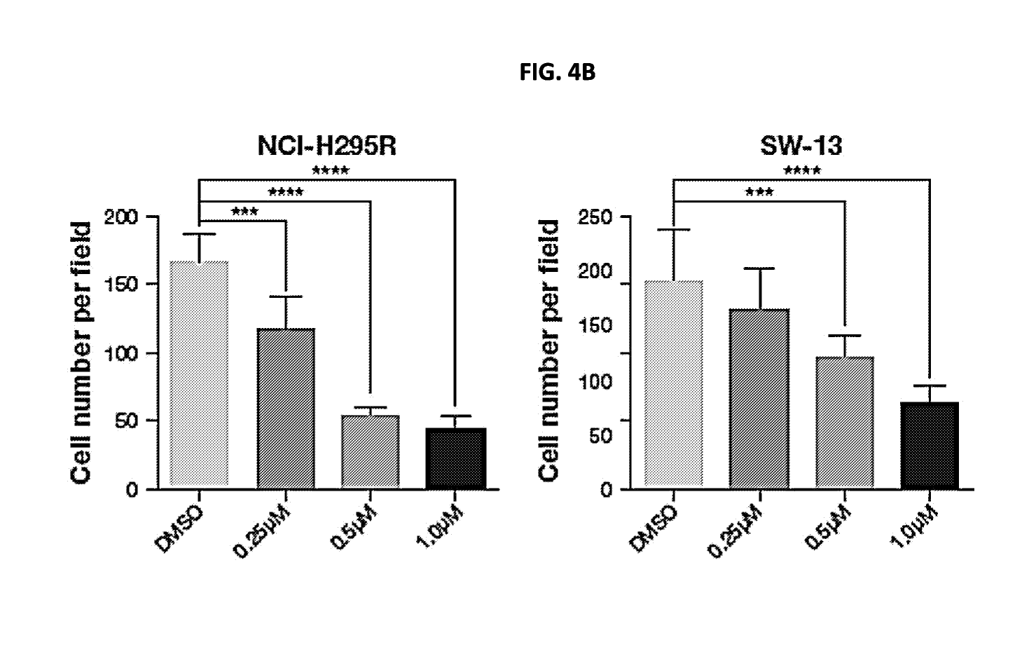

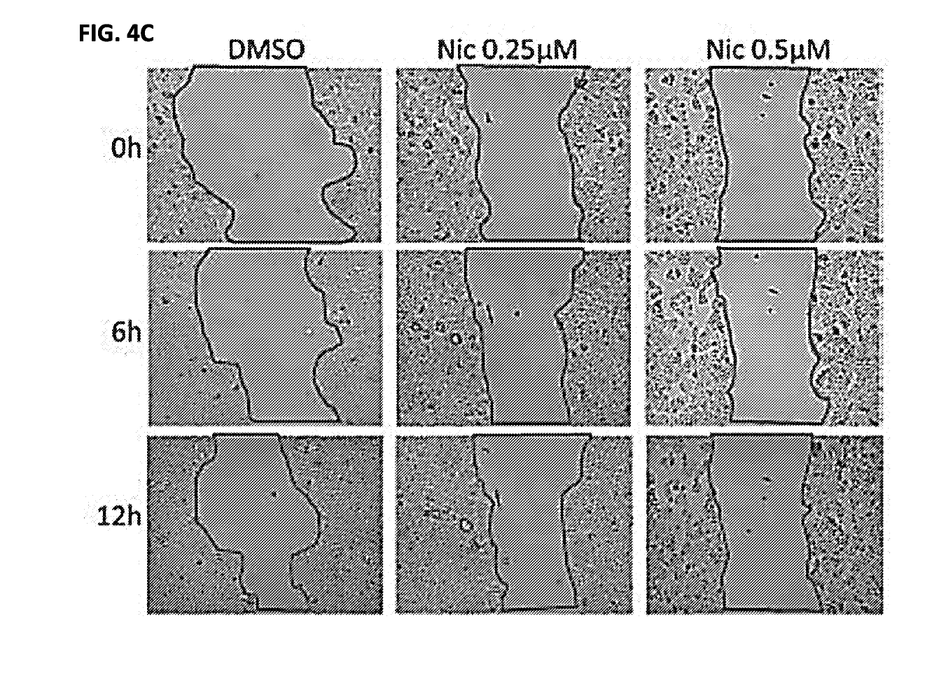

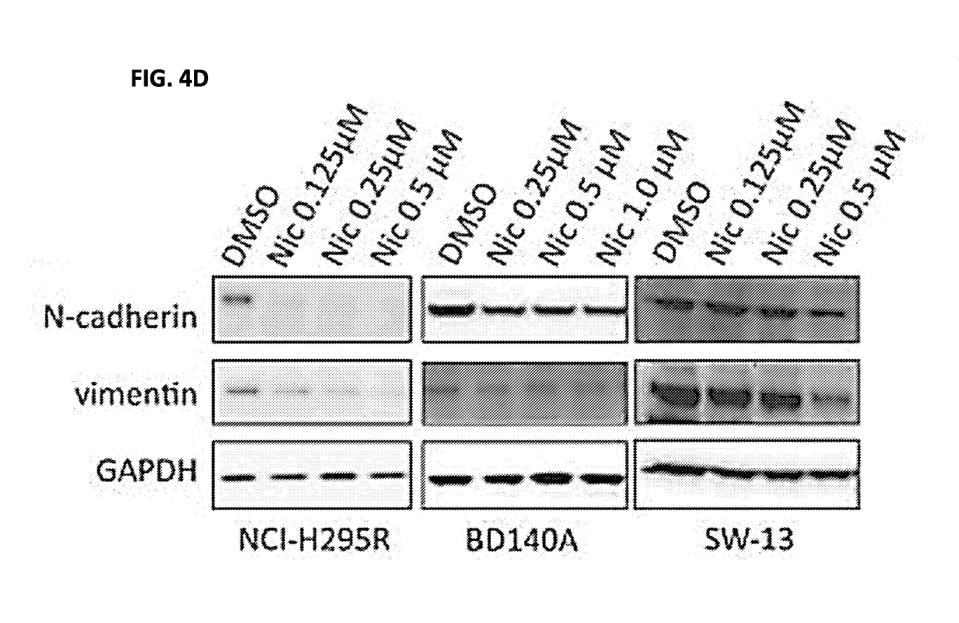

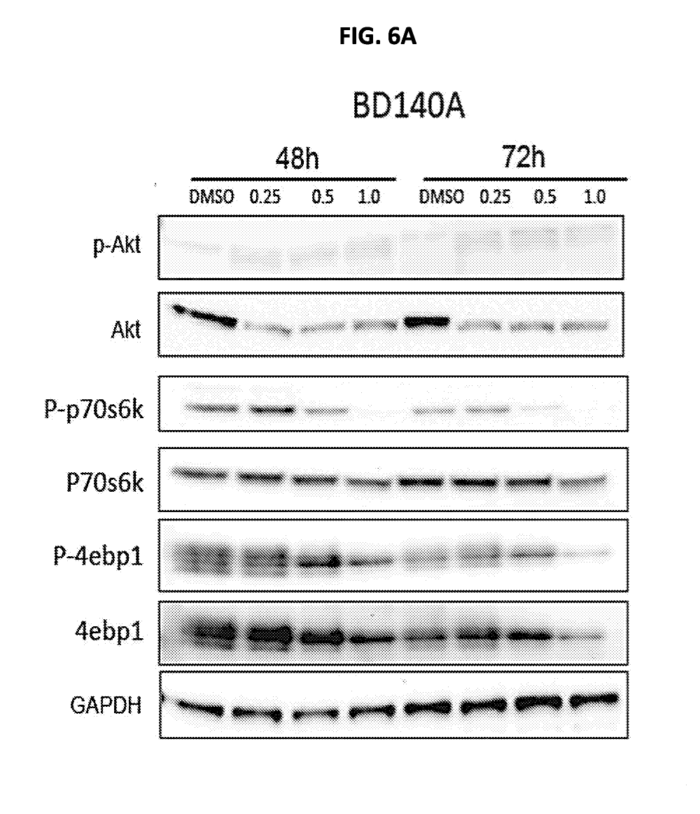

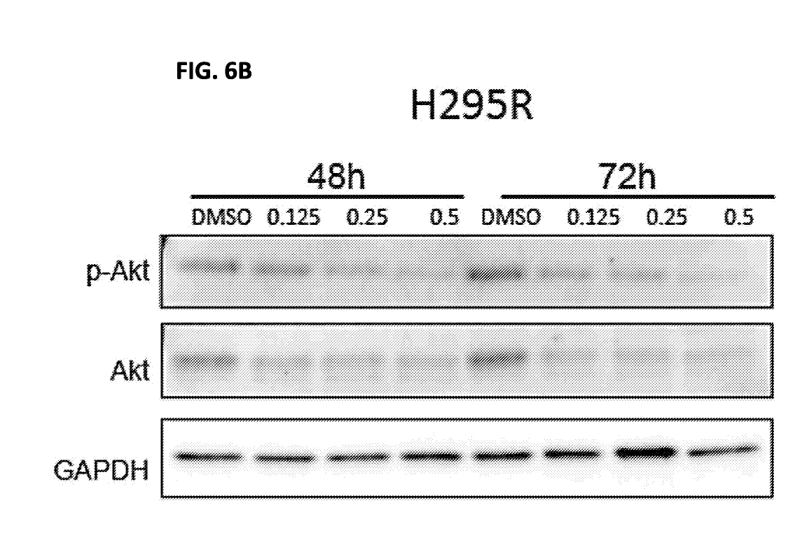

The effect of niclosamide on β-catenin, a pathway altered in over 30% of ACC cases, was examined. Niclosamide reduced the expression of β-catenin in all three cell lines (FIG. 4A). Niclosamide also inhibited the AKT pathway in BD140A and H295R cells (FIGS. 6A and 6B). Reduced cellular migration with niclosamide treatment was found in transwell migration assays (FIG. 4B). Niclosamide's effect on migration in BD140A cells was assessed through a wound-heal assay because BD140A cells do not migrate in the Boyden chamber model. Niclosamide treatment decreased BD140A cell migration after 6 hours and 12 hours of treatment, as compared to the vehicle control (FIG. 4C). Given the observed effect of niclosamide on migration, whether niclosamide altered the level of EMT mediators was evaluated. Niclosamide reduced the expression of N-cadherin and vimentin (FIG. 4D).

### EXAMPLE 6

### Niclosamide Inhibits ACC Tumor Growth in Vivo

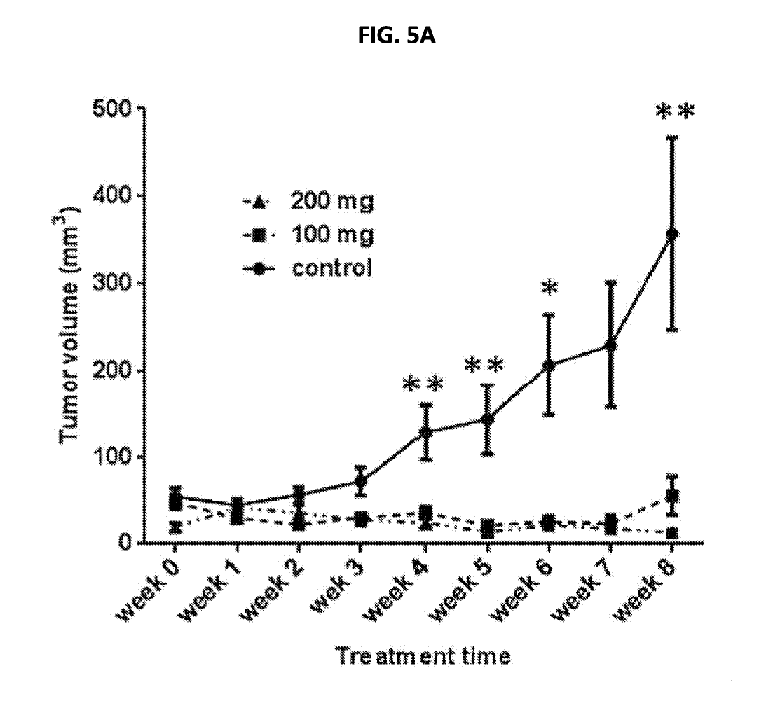

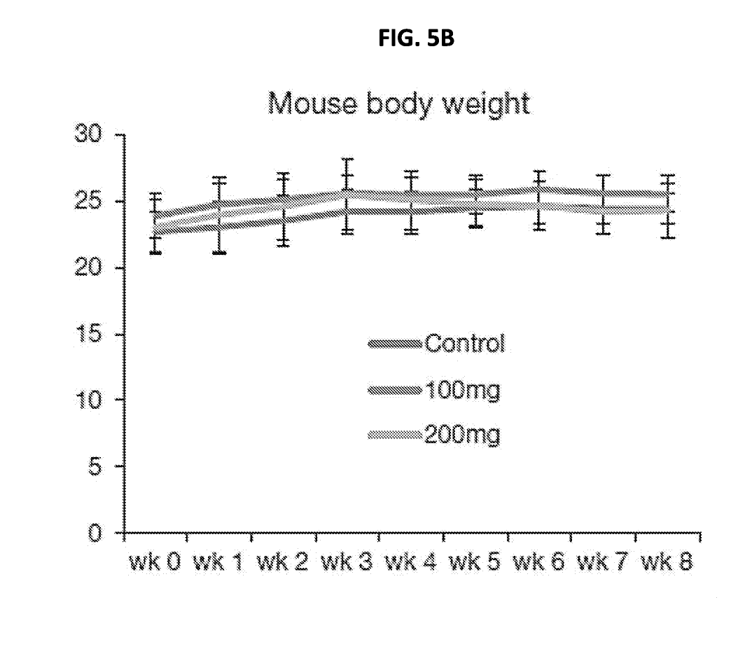

To confirm the in vitro observations above, the effect of niclosamide treatment was evaluated in ACC xenografts. Niclosamide treatments, at both doses (100 mg/kg and 200 mg/kg), were well tolerated, with no observed toxicity or side effects in the mice. There were no significant weight differences among the groups (FIG. 5). Four weeks after treatment, mice treated with niclosamide at 100 mg/kg and 200 mg/kg showed a 60% and 80% inhibition in tumor growth, respectively, as compared to the vehicle control group (P<0.01 for both groups) (FIG. 5). The same treatment schedule was maintained for 8 weeks, at which time, more than 90% tumor growth inhibition was observed for the two treated groups, as compared to the control group.

### EXAMPLE 7

### Synergistic Effect of Niclosamide and Mitotane

This example describes a method used to evaluate proliferation of H2965R adrenocortical carcinoma cells in the presence of (1) niclosamide alone (at 0.05 μM, 0.075 μM, 0.1 μM, 0.15 μM, or 0.2 μM), (2) mitotane alone (at 0.5 μM, 0.75 μM, 1 μM, 1.5 μM, or 2 μM), or (3) a combination of both niclosamide and mitotane. Although particular methods, dosages, and modes of administrations are provided, one skilled in the art will appreciate that certain variations can be made without substantially affecting the treatment.

NCI-H295R cells were cultured in Dulbecco modified Eagle medium (DMEM) supplemented with 2.5% NuSerum (BD Biosciences, San Jose, Calif.) and 0.1% ITS premix (BD Biosciences, San Jose, Calif.). Cells were maintained in 5% CO₂ atmosphere at 37° C. 3×10³ cells were plated in 96-well plates. 100 μL fresh culture medium containing single drug (mitotane, niclosamide), in combination (mitotane and niclosamide) or vehicle (with appropriate % volume DMSO used to solubilize the drugs) was added. Cell count was determined using the CyQuant kit (Life technologies, Grand Island, N.Y.) according to the manufacturer's instructions, and absorbance (cell number) was measured using a SpectraMax M5 microplate reader (Molecular Devices, Sunnyvale, Calif., ex485/em538). Assays were performed in quadruplicate and the experiments were repeated three times.

Statistical analysis was performed using GraphPad Prism 6 software (GraphPad Software, La Jolla, Calif.). Data was analyzed using a two-tailed Mann-Whitney test. Statistical significance was defined as a p-value of less than 0.05. Data are presented as mean±standard deviation (SD). To determine if combination treatment with niclosamide and mitotane had an additive, synergistic or antagonistic effect on cellular proliferation inhibition, the combination index (CI) was calculated based on the Chou-Talalay method (Chou, Cancer Res, 2010. 70(2):440-6) . Defined as a CI index of <1=synergistic, 1=additive, and >1 antagonistic.

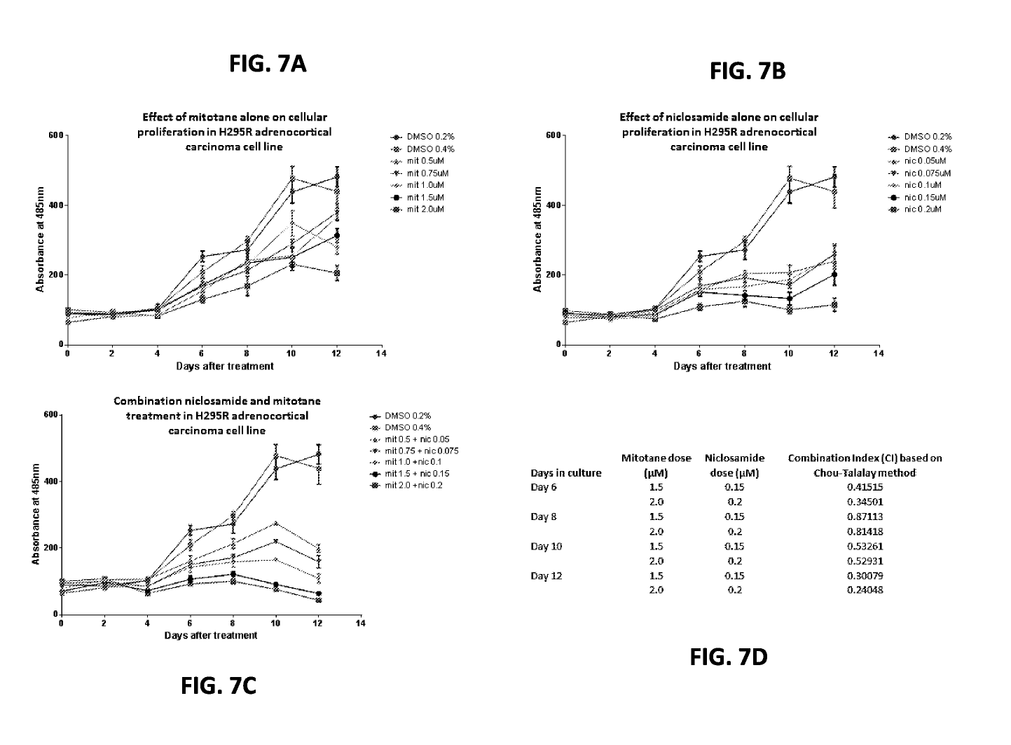

As shown in FIGS. 7A and 7B, both mitotane and niclosamide alone had some effect on reducing proliferation of H295R cells. However, as shown in FIGS. 7C and 7D, when these reagents were combined, a synergistic effect was achieved.

### EXAMPLE 8

### Method to Treat ACC

This example describes an exemplary method that can be used to treat ACC in humans by administering niclosamide either alone or in combination with other therapeutic agents, such as additional agents listed in Table 1. Although particular methods, dosages, and modes of administrations are provided, one skilled in the art will appreciate that certain variations can be made without substantially affecting the treatment.

Based upon the teaching disclosed herein, ACC, such as locally advanced and metastatic ACC can be treated by administering a therapeutically effective amount of niclosamide alone or in combination with other therapeutic agents, thereby reducing or eliminating one or more signs or symptoms associated with the ACC.

Briefly, the method can include screening subjects to determine if they have a ACC. Subjects having ACC are selected. In one example, subjects having ACC which has been non-responsive to standard ACC therapies are selected. In one example, a clinical trial would include half of the subjects following the established protocol for treatment of ACC (such as a normal chemotherapy/radiotherapy/surgery regimen). The other half would follow the established protocol for treatment of the ACC (such as a normal chemotherapy/radiotherapy/surgery regimen) in combination with administration of the therapeutic compositions described above. In some examples, the tumor is surgically excised (in whole or part) prior to treatment with the therapeutic compositions. In another example, a clinical trial would include half of the subjects following the established protocol for treatment of ACC (such as a normal chemotherapy/radiotherapy/surgery regimen). The other half would follow the administration of the therapeutic compositions described above. In some examples, the tumor is surgically excised (in whole or part) prior to treatment with the therapeutic compositions.

### Screening Subjects

In some examples, the subject is first screened to determine if they have ACC. In particular examples, the subject is screened to determine if the adrenocortical tumor is malignant, indicating ACC, or benign. Examples of methods that can be used to screening for ACC include a combination of ultrasound, tissue biopsy, and serum blood levels. If blood or a fraction thereof (such as serum) is used, 1-100 μl of blood is collected. Serum can either be used directly or fractionated using filter cut-offs to remove high molecular weight proteins. If desired, the serum can be frozen and thawed before use. If a tissue biopsy sample is used, 1-100 μg of tissue is obtained, for example using a fine needle aspirate. The biological sample (e.g., tissue biopsy or serum) is analyzed to determine expression of ACC biomarkers. Expression ACC biomarkers, such as miR-483-3p or miR-483-5p and IGF2-mRNA, indicates that the tumor is malignant and further that it can be treated with the disclosed therapies.

In a specific example, a tissue biopsy is procured from the adrenal cortex. RNA is isolated and purified from these cells using routine methods, such as using the methods described in Example 1. Alterations in expression levels of ACC biomarkers are determined by performing real-time PCR or microarray analysis. Detection of ACC biomarkers associated with ACC is indicative that the subject has ACC and is a candidate for receiving the therapeutic compositions disclosed herein. However, such pre-screening is not required prior to administration of the therapeutic compositions disclosed herein (such as niclosamide).

### Pre-treatment of Subjects

In particular examples, the subject is treated prior to administration of a therapeutic composition that includes one or more agents provided in Table 1, including niclosamide alone or in combination with such agents. However, such pre-treatment is not always required, and can be determined by a skilled clinician. For example, the tumor can be surgically excised (in total or in part) prior to administration of the therapy. In addition, the subject can be treated with an established protocol for treatment of the particular tumor present (such as a normal chemotherapy/radiotherapy regimen).

### Administration of Therapeutic Compositions

Following subject selection, a therapeutic effective dose of the composition is administered to the subject, wherein the composition includes one or more agents listed in Table 1, such as niclosamide. Niclosamide is administered in vivo by oral administration at 100 mg/kg to 400 mg/kg each day. Administration of the therapeutic compositions can occur in combination with other ACC treatments or can be continued after chemotherapy and radiation therapy is stopped and can be taken long term (for example over a period of weeks, months or years).

### Assessment

Following the administration of one or more therapies, subjects having a malignant tumor (for example ACC) can be monitored for tumor treatment, such as regression or reduction in metastatic lesions, tumor growth or vascularization. In particular examples, subjects are analyzed one or more times, starting 7 days following treatment. Subjects can be monitored using any method known in the art. For example, diagnostic imaging can be used (such as x-rays, CT scans, MRIs, ultrasound, fiber optic examination, and laparoscopic examination), as well as analysis of biological samples from the subject (for example analysis of blood, tissue biopsy, or other biological samples), such as analysis of the type of cells present, or analysis for a particular tumor marker. In one example, if the subject has advanced ACC, assessment can be made using ultrasound, MRI, or CAT scans, or analysis of the type of cells contained in a tissue biopsy. It is also contemplated that subjects can be monitored for the response of their tumor(s) to therapy during therapeutic treatment by at least the aforementioned methods.

### Additional Treatments

In particular examples, if subjects are stable or have a minor, mixed or partial response to treatment, they can be re-treated after re-evaluation with the same schedule and preparation of agents that they previously received for the desired amount of time, such as up to a year of total therapy. A partial response is a reduction in size or growth of some tumors, but an increase in others.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

1. A method of treating a malignant adrenocortical tumor in a subject, comprising:
administering to the subject with a malignant adrenocortical tumor an effective amount of one or more agents listed in Table 1 to reduce one or more symptoms associated with the malignant adrenocortical tumor, thereby treating the malignant adrenocortical tumor.

2. The method of claim 1, wherein the one or more agents from Table 1 comprise niclosamide.

3. The method of claim 2, wherein the method further comprises administering to the subject an effective amount of:
mitotane;
cisplatin, doxorubicin, etoposide, and mitotane; or
streptozotocin and mitotane.

4. The method of claim 2, wherein the one or more agents from Table 1 further comprise dactinomycin, emetine, ouabain, omacetaxine, idarubicin, aclarubicin, or combinations thereof.

5. The method of claim 2, wherein the one or more agents from Table 1 further comprise aclarubicin, carminomycin, dactinomycin, idarubicin, omacetaxine mepesuccinate, plicamycin, trabectedin, or combinations thereof.

6. (canceled)

7. The method of claim 1, wherein the one or more agents comprise 4-chloromercuriphenol, alpha-tomatine, auranofin, chromomycin A3, deslano side, digitoxin, digoxin, lanatoside A or C, o-(chloro)-mercuriphenol, zinc pyrithione, or combinations thereof.

8. The method of claim 1, wherein administering comprises oral administration.

9. The method of claim 8, wherein 100 mg/kg to 300 mg/kg of the one or more agents listed in Table 1 are orally administered.

10. The method of any one of claim 1, wherein the malignant adrenocortical tumor is adrenocortical carcinoma (ACC).

11. The method of claim 1, wherein the malignant adrenocortical tumor is locally advanced and metastatic ACC.

12. The method of claim 1, further comprising selecting a subject with a malignant adrenocortical tumor.

13. The method of claim 12, wherein selecting a subject with a malignant adrenocortical tumor comprises selecting a subject non-responsive to standard therapy malignant adrenocortical tumor therapy.

14. The method of claim 13, wherein the standard therapy comprises administration of
mitotane;
cisplatin, doxorubicin, etoposide, and mitotane; or
streptozotocin and mitotane.

15. The method of claim 1, wherein reducing one or more symptoms associated with the malignant adrenocortical tumor comprises inhibiting tumor growth.

16. The method of claim 15, wherein tumor growth is inhibited
by at least 60% as compared to tumor growth prior to administering the effective amount of one or more agents listed in Table 1;
by 60% to 80% as compared to tumor growth prior to administering the effective amount of one or more agents listed in Table 1; or
by at least 90% as compared to tumor growth prior to administering the effective amount of one or more agents listed in Table 1.

17. The method claim 1, further comprising administering an additional therapeutic agent, prior to, concurrent with, or subsequent to, administering the effective amount of one or more agents listed in Table 1.

18. The method of claim 17, wherein the additional therapeutic agent is a chemotherapeutic agent.

19. The method of claim 18, wherein the chemotherapeutic agent is cisplatin, doxorubicin, etoposide, mitotane, streptozocin, or a combination thereof.

20. The method of claim 1, wherein administering an effective amount of one or more agents listed in Table 1 comprises administering a therapeutically effective amount of the one or more agents listed in Table 1 with a pharmaceutically acceptable carrier.

21. The method of claim 1, wherein the subject is a human.
